SEQUENCE LISTING (4 sequences)

SEQ ID NO: 1 - protein, 7 aa, Artificial Sequence
  Designed peptide
YLYEIAR

SEQ ID NO: 2 - protein, 7 aa, Artificial Sequence
  Designed peptide
YLFEIAR

SEQ ID NO: 3 - protein, 7 aa, Artificial Sequence
  Designed peptide
FLYEIAR

SEQ ID NO: 4 - protein, 7 aa, Artificial Sequence
  Designed peptide
FLFEIAR

(19) United States
(12) Patent Application Publication
(10) Pub. No.: US 2010/0210564 A1
(43) Pub. Date: Aug. 19, 2010

(54) DRUG OR FOOD CONTAINING A PEPTIDE

(75) Inventors: Kousaku OHINATA, Uji-shi (JP); Norimasa KANEGAWA, Uji-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignees: Kyoto University, Kyoto-shi (JP); Kyowa Hakko Bio Co., Ltd., Tokyo (JP)

(21) Appl. No.: 12/697,694

(22) Filed: Feb. 1, 2010

(30) Foreign Application Priority Data
Feb. 2, 2009 (JP) 2009-21958

(51) Int. Cl. A61K 38/08; C07K 5/08; C07K 5/06; C07K 7/06; A61P 25/18; A61P 25/24; A61K 38/05; A61K 38/06; A61P 25/20

(52) U.S. Cl. 514/16; 530/331; 562/445; 530/329; 548/491; 548/338.1; 514/19; 514/18

(57) ABSTRACT

A pharmaceutical or a foodstuff including as an active ingredient a peptide containing Tyr (Y), Phe (F), Trp (W), or His (H) and a hydrophobic amino acid adjacent thereto, or an analog thereof.

### BACKGROUND OF THE INVENTION

(1) Field of the Invention

The present invention relates to pharmaceuticals and pharmaceutical compositions that act on the nervous system. The present invention further relates to peptides and analogs thereof; and more specifically, to peptides and analogs thereof that activate at least one of 5-HT_{1A} receptor, D₁ receptor, and GABA_{A} receptor. The present invention further relates to anxiolytic or sleep-enhancing foodstuffs.

(2) Description of the Related Art

Reflecting today's stressful society, the increasing number of individuals with psychiatric disorders such as anxiety disorders, schizophrenia, and depression has been a problem. Anxiety per se is inherently necessary as a warning to help living organisms avoid danger; however, it is known that excessive anxiety involves the onset or the progression of symptoms of psychiatric disorders as mentioned above, and also increases the risk of the onset of lifestyle-related diseases. Therefore, foodstuffs and pharmaceuticals for relieving mental stress are expected to develop. Desirably, compounds having such anxiolytic effects can be manufactured at low cost, and are effective by oral administration.

Dipeptides are relatively easy to manufacture, and can also be produced in large quantities from enzymatic digests of food proteins. In practice, ACE inhibitor peptides that reduce blood pressure, and aspartame, which is an intensive sweetener, are used as foodstuffs. However, there has been no report on a dipeptide having anxiolytic effects. Opioid peptides having analgesic effects are known as bioactive oligopeptides. Furthermore, soymorphin derived from a major soy protein, β-conglycinin, has been revealed to have anxiolytic effects (Japanese Unexamined Patent Publication No. 2007-91656).

### SUMMARY OF THE INVENTION

An object of the present invention is to provide pharmaceuticals and foodstuffs having anxiolytic, sedative, sleep-enhancing, and like effects, and having few or no side effects.

The inventors investigated various peptides for anxiolytic effects in order to solve this problem, and consequently found that specific short-chain peptides such as Tyr-Leu, Phe-Leu, Trp-Leu, His-Leu, Ile-Tyr, Leu-Trp, and the like have potent anxiolytic, sedative, and like effects, and have the effect of activating at least one receptor selected from the group consisting of 5-HT_{1A} receptor, D₁ receptor, and GABA_{A} receptor, thereby accomplishing the invention.

The present invention provides pharmaceuticals; anxiolytic or sleep-enhancing foodstuffs; and a method for relieving anxiety or enhancing sleep, as given below.

Item 1. A pharmaceutical or a pharmaceutical composition comprising, as an active ingredient, a peptide containing Tyr, hereinafter also abbreviated as Y, Phe, hereinafter also abbreviated as F, Trp, hereinafter also abbreviated as W, or His, hereinafter also abbreviated as H, and a hydrophobic amino acid adjacent thereto, or an analog thereof.

Item 2. The pharmaceutical or the pharmaceutical composition according to Item 1, wherein the active ingredient is a peptide containing Tyr or Phe and a hydrophobic amino acid adjacent thereto, or an analog thereof.

Item 3. The pharmaceutical or the pharmaceutical composition according to Item 1, wherein the active ingredient is YL, FL, WL, HL, YI, FI, YV, LY, LF, LW, IY, IF, (Y/F/W/H)L(Y/F/W), (Y/F/W/H)LQ, L(Y/F/W)L, or (Y/F/W/H)L(Y/F/W)EIAR, wherein, hereinafter, L represents Leu, I represents Ile, V represents Val, Q represents Gln, E represents Glu, A represents Ala, R represents Arg, (Y/F/W/H) represents H, W, Y, or F, and (Y/F/W) represents W, F, or Y.

Item 4. The pharmaceutical or the pharmaceutical composition according to Item 3, wherein the active ingredient is YL, FL, WL, HL, YI, FI, FV, LY, LF, LW, IY, IF, YLY, YLQ, LYL, or YLYEIAR.

Item 5. The pharmaceutical or the pharmaceutical composition according to any one of Items 1 to 4, which is an anxiolytic drug, a sleep-inducing drug, a sleep-enhancing drug, a drug for treating schizophrenia, or an antidepressant drug.

Item 6. An anxiolytic or sleep-enhancing foodstuff comprising a peptide containing Tyr, Phe, Trp, or His and a hydrophobic amino acid adjacent thereto, or an analog thereof.

Item 7. The anxiolytic or sleep-enhancing foodstuff according to Item 6, comprising a peptide containing Tyr or Phe and a hydrophobic amino acid adjacent thereto, or an analog thereof.

Item 8. The anxiolytic or sleep-enhancing foodstuff according to Item 6, comprising YL, FL, WL, HL, YI, FI, YV, LY, LF, LW, IY, IF, (Y/F/W/H)L(Y/F/W), (Y/F/W/H)LQ, L(Y/F/W)L, or (Y/F/W/H)L(Y/F/W)EIAR.

Item 9. The anxiolytic or sleep-enhancing foodstuff according to Item 8, comprising YL, FL, WL, HL, YI, FI, FV, LY, LF, LW, IY, IF, YLY, YLQ, LYL, or YLYEIAR.

Item 10. A method for relieving anxiety or enhancing sleep, comprising administering an effective amount of a peptide containing Tyr, Phe, Trp, or His and a hydrophobic amino acid adjacent thereto, or an analog thereof, to a subject in need thereof.

Anxiolytic drugs, drugs for treating sleep disorders, drugs for treating schizophrenia, antidepressant drugs, and drugs for preventing these diseases, containing as active ingredients the peptides of the invention or analogs thereof, have few side effects and are suitable for long-term use.

Furthermore, the drugs of the invention are effective by oral administration.

Furthermore, natural short-chain peptides can be ingested as foods; therefore, when ingested as foods by individuals who are not ill but who have anxious tendencies or trouble sleeping, such peptides can be expected to prevent the above-mentioned diseases in such individuals.

The peptides of the invention and analogs thereof have the effect of activating at least one receptor selected from the group consisting of 5-HT_{1A} receptor, D₁ receptor, and GABA_{A} receptor, and are thus expected to have the effect of preventing or treating various diseases attributed to the effect of activating these receptors.

Furthermore, the peptides of the invention and analogs thereof are free of side effects caused by the activation of opioid receptor agonists.

### BRIEF DESCRIPTION OF THE DRAWINGS

FIG. 1 shows an elevated plus-maze;

FIG. 2A shows the anxiolytic effects of i.p.-administered YL (Ex. 1);

FIG. 2B shows the anxiolytic effects of p.o.-administered YL (Ex. 1);

FIG. 3 shows the anxiolytic effects of i.p.-administered FL (Ex. 2);

FIG. 4 shows the anxiolytic effect of i.p.-administered YI (Ex. 3);

FIG. 5 shows the anxiolytic effect of i.p.-administered FI (Ex. 4);

FIG. 6 shows the anxiolytic effect of i.p.-administered LY (Ex. 5);

FIG. 7 shows the anxiolytic effect of i.p.-administered LF (Ex. 6);

FIG. 8 shows the anxiolytic effect of i.p.-administered IY (Ex. 7);

FIG. 9 shows the anxiolytic effect of i.p.-administered IF (Ex. 8);

FIG. 10 shows the anxiolytic effect of i.p.-administered YLY (Ex. 9);

FIG. 11 shows the anxiolytic effect of i.p.-administered YLQ (Ex. 10);

FIG. 12 shows the anxiolytic effect of i.p. administered LYL (Ex. 11);

FIG. 13 shows the anxiolytic effect of i.p.-administered YLYEIAR (Ex. 12);

FIG. 14 shows the anxiolytic effect of i.p.-administered Y and Y&L (Comp. Ex. 1 and 2);

FIG. 15 shows the effect of a 5HT_{1A} receptor antagonist on the anxiolytic activity of YL;

FIG. 16 shows the effect of a dopamine D₁ receptor antagonist on the anxiolytic activity of YL;

FIG. 17 shows the effect of a GABA_{A} receptor antagonist on the anxiolytic activity of YL;

FIG. 18 shows the effect of a benzodiazepine site antagonist on the anxiolytic activity of YL;

FIG. 19 shows the effect of a μ-opioid receptor antagonist on the anxiolytic activity of YL;

FIG. 20 shows the effect of antagonists for the δ-opioid receptor and σ₁ receptor on the anxiolytic activity of YL;

FIG. 21 shows the effect of a cyclooxygenase inhibitor on the anxiolytic activity of YL;

FIG. 22 shows the anxiolytic effect of i.p-administered YV (Ex. 13);

FIG. 23 shows the anxiolytic effect of i.p.-administered WL (Ex. 14);

FIG. 24 shows the anxiolytic effect of i.p.-administered LW (Ex. 15);

FIG. 25 shows the anxiolytic effects of p.o.-administered WL and LW (Ex. 14 and 15);

FIG. 26 shows the anxiolytic effect of i.p.-administered HL (Ex. 16);

FIG. 27 shows a comparison of the anxiolytic effects between YL and diazepam (Comp. Ex. 3); and

FIG. 28 shows a presumed anxiolytic mechanism of YL.

### DESCRIPTION OF THE PREFERRED EMBODIMENTS

### Pharmacological Effects

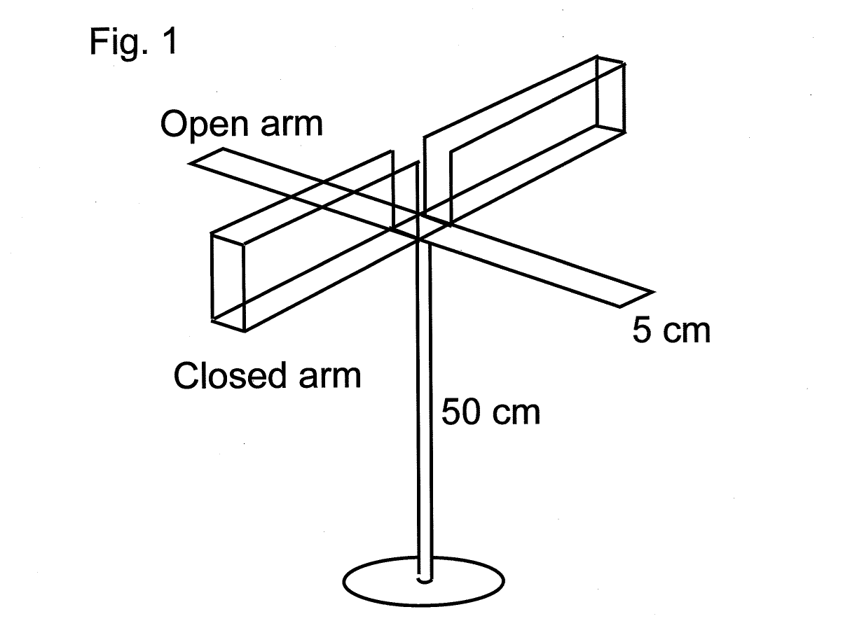

In the present invention, the anxiolytic effects can be evaluated by the elevated plus-maze test, developed and widely used as a method for evaluating anxiety-related behaviors to screen anxiolytic drugs (FIG. 1). Specifically, the test is performed as follows: a candidate substance for an anxiolytic drug is orally or intraperitoneally administered to a mouse; 30 minutes after the administration, the mouse is placed in an elevated plus-maze; and the potency of the anxiolytic effects is evaluated based on the number of entries to the open arms and a change in the times spent on the open arms.

Although the anxiolytic effects of the peptides of the invention are blocked by the 5-HT_{1A} receptor antagonist WAY100135, the peptides do not have affinity for 5-HT_{1A} receptor. This confirms that the anxiolytic effects of the peptides are mediated by the activation of 5-HT₃ₐ receptor, i.e., the peptides have effects that are similar to those of 5-HT₁ₐ receptor agonists or partial agonists. It is presumed that endogenous serotonin release is being promoted. The peptides of the invention are presumed to have the effect of preventing or treating depression, schizophrenia, and like diseases, enhancing memory, etc. by activating 5-HT_{1A} receptor. The pharmaceuticals and pharmaceutical compositions of the invention are also useful as 5-HT_{1A} receptor agonists, drugs for preventing or treating depression, drugs for preventing or treating schizophrenia, memory-enhancing drugs, anxiolytic drugs, sleep-enhancing drugs, etc.

Although the anxiolytic effects of the peptides of the invention are blocked by the dopamine D₁ receptor antagonist SCH23390, the peptides do not have affinity for dopamine D₁ receptor. This confirms that the anxiolytic effects of the peptides are mediated by the activation of dopamine D₁ receptor, i.e., the peptides have effects that are similar to those of D₁ receptor agonists or partial agonists. It is presumed that endogenous dopamine release is being promoted. Given that the spatial perception performance is mediated by dopamine D₁ receptor, and the function of D₁ receptor is decreased in schizophrenic individuals, the peptides of the invention can be expected to prevent or treat diseases such as dementia, schizophrenia, and the like.

Although the anxiolytic effects of the peptides of the invention are blocked by GABA_{A} receptor antagonist (bicuculline), the peptides do not have affinity for GABA_{A} receptor. This confirms that the effects of the peptides are mediated by GABA_{A} receptor, i.e., the peptides have effects that are similar to those of GABA_{A} receptor agonists or partial agonists. It is presumed that endogenous GABA release is being promoted. Furthermore, the anxiolytic effects of the peptides of the invention are significantly blocked by the benzodiazepine receptor antagonist flumazenil. This confirms that the anxiolytic effects are partially mediated by the benzodiazepine site of GABA_{A} receptor. Given that GABA_{A} receptor is known to have sleep-inducing effects, the peptides of the invention and analogs thereof are considered to have sleep-inducing effects in addition to anxiolytic effects, and are also useful as sleep-inducing drugs. In particular, LF has been shown to reduce locomotor activity, and is therefore useful as a sleep-inducing drug.

The inventors previously found that soymorphin, a μ-opioid peptide derived from the primary soy protein β-conglycinin, shows anxiolytic effects mediated by μ-opioid receptor (Japanese Unexamined Patent Publication No. 2007-91656). The anxiolytic effects of the peptides of the invention are not blocked by the g-opioid receptor antagonist naloxone. This confirms that the anxiolytic effects of the peptides are not mediated by the g-opioid receptors, unlike the peptides disclosed in Japanese Unexamined Patent Publication No. 2007-91656.

It is known that δ-opioid rubiscolin derived from Rubisco, a major protein from green leaves, demonstrates anxiolytic effects by directly acting on the δ-opioid receptor, and subsequently activating the σ₁ receptor. The anxiolytic effects of the peptides of the invention are not blocked by the δ-opioid receptor antagonist naltrindole. This confirms that the anxiolytic effects of the peptides are not mediated by the δ-opioid receptors. Additionally, the anxiolytic effects of the peptides of the invention are not blocked by the α₁ receptor antagonist BMY14802. This confirms that the anxiolytic effects of the peptides are not mediated by the σ₁ receptor.

Rubimetide (MRW), which is another anxiolytic peptide derived from Rubisco, shows anxiolytic effects by promoting the release of prostaglandin D₂, and activating DP₁ receptor of the two types of PGD₂ receptors. The anxiolytic effects of the peptides of the invention are not blocked by the cyclooxygenase inhibitor indomethacin. This confirms that the anxiolytic effects of the peptides are not associated with the prostaglandin system. Therefore, the peptides of the invention are considered to have a mechanism of action completely different from those of the previous anxiolytic peptides.

Preferred active ingredients in the invention have been confirmed to be effective by oral administration.

### Active Ingredient

The active ingredient of the anxiolytic drug of the invention is a peptide or an analog thereof.

The peptide contains 2 to 8, preferably 2 to 7, more preferably 2 to 6, still more preferably 2 to 5, particularly preferably 2 to 4, and most preferably 2 to 3 amino acids; and has Y (Tyr), F (Phe), W (Trp), or H (His), preferably Y (Tyr), F (Phe), or W (Trp), and more preferably Y (Tyr) or W (Trp).

Each of the amino acids forming the peptide may be an L-amino acid, D-amino acid, or DL-amino acid (the mixture of D- and L-amino acids includes both of a racemic amino acid and an amino acid containing an excess of one enantiomer). Preferably, the peptide contains L-amino acids or D-amino acids solely. A peptide containing L-amino acids solely is preferred.

When the peptide used in the invention has two or more asymmetric carbons, it may be in the form of each enantiomer, a diastereomer, or a mixture thereof with a given ratio. Separation of the enantiomer or diastereomer may be performed on a general column, for example, on a chiral column, using a method in which the enantiomer or diastereomer is optically resolved in the form of a derivative by introducing an optically active group, and the optically active group is subsequently removed; or a method in which the enantiomer or diastereomer is optically resolved by forming a salt with an optically active acid or base; or any other known method.

Examples of salts of the peptide or analog thereof include acid addition salts and base salts. Examples of acid addition salts include salts with inorganic acids such as hydrochloric acid, sulfuric acid, nitric acid, phosphoric acid, hydrobromic acid, perchloric acid, and the like; and salts with organic acids such as citric acid, succinic acid, maleic acid, fumaric acid, malic acid, tartaric acid, p-toluenesulfonic acid, benzenesulfonic acid, methanesulfonic acid, trifluoroacetic acid, and the like. Examples of base salts include salts with bases such as alkali metal salts with, e.g., sodium, potassium, and lithium; and alkaline earth metal salts with, e.g., calcium and magnesium.

Examples of solvates include solvates with water (hydrates), methanol, ethanol, isopropanol, acetic acid, tetrahydrofuran, acetone, dimethylformamide, dimethyl sulfoxide, dimethylacetamide, acetamide, ethylene glycol, propylene glycol, and dimethoxyethane.

Cases where the Active Ingredient is a Peptide

In preferred embodiments of the invention, amino acids forming the active ingredient peptide contain at least two types of amino acids; one being Y (Tyr), F (Phe), W (Trp), or H (His), and the other being a hydrophobic amino acid selected from the group consisting of L (Leu), I (Ile), V (Val), norleucine (Nle), and norvaline (Nva). These two types of amino acids form adjacent units, wherein H, W, Y, or F may be at the N-terminus of the peptide, as in HL, WL, YL, FL, YV, FV, (Y/F/W/H)-norleucine, or (Y/F/W/H)-norvaline; or wherein H, W, Y, or F may be at the C-terminus of the peptide, as in LY, LF, LW, LH, IY, IF, IW, IH, VH, VW, VY, VF, norleucine-(Y/F/W/H), or norvaline-(Y/F/W/H). At the N-terminus or C-terminus of such units of two types of amino acids, 1 to 6, 1 to 5, 1 to 4, 1 to 3, 1 or 2, or 1 amino acid(s) may be further linked via peptide bonds.

Y/F/W/H means Y, F, W, or H, and may be a peptide wherein two or more (preferably two) amino acids selected from Y, F, W, H are coupled, such as HW, HY, HF, WH, YH, FH, WY, WF, YW, FW, HH, WW, YF, FY, YY, FF, and the like. Y/F means Y or F, and may be a peptide wherein two or more (preferably two) amino acids selected from Y and F, such as YF, FY, YY, FF, and the like.

Instead of H, W, Y, or F, 3,4-dihydroxyphenylalanine (DOPA), 4-methoxyphenylalanine, 4-mercaptophenylalanine, and the like are also usable.

In the peptide of the invention, Y, F, W, or H is preferably present at the N-terminus.

The following are peptides having anxiolytic effects that can be preferably used in the invention:

Y-(hydrophobic amino acid);

F-(hydrophobic amino acid);

W-(hydrophobic amino acid);

H-(hydrophobic amino acid);

(hydrophobic amino acid)-H;

(hydrophobic amino acid)-W;

(hydrophobic amino acid)-Y;

(hydrophobic amino acid)-F;

(Y/F/W/H)-L-(given amino acid)ₙ;

(given amino acid)ₙ-(Y/F/W/H)-L;

(given amino acid)ₙ₁-(Y/F/W/H)-L-(given amino acid)ₙ₂;

(hydrophobic amino acid)-(Y/F/W/H)-(hydrophobic amino acid);

(Y/F/W/H)-(hydrophobic amino acid)-(Y/F/W/H);

{(Y/F/W/H)-(hydrophobic amino acid)}ₘ;

{(hydrophobic amino acid)-(Y/F/W/H)}ₚ;

{(Y/F/W/H)-(hydrophobic amino acid)}_{q}-Y; and

{(hydrophobic amino acid)-(Y/F/W/H)}ᵣ-(hydrophobic amino acid); wherein

the hydrophobic amino acid or hydrophobic amino acids are each independently selected from the group consisting of L (Leu), I (Ile), V (Val), norleucine (Nle), and norvaline (Nva), each being preferably L (Leu), I (Ile), or V (Val), more preferably L (Leu) or I (Ile), and particularly preferably L(Leu).

The given amino acid is an amino acid selected from the group consisting of 20 natural amino acids, i.e., Leu, Ile, Val, Ala, Gly, Met, Ser, Cys, His, Asn, Asp, Glu, Gln, Thr, Lys, Trp, Phe, Arg, Tyr, and Pro; and β-alanine, sarcosine, ornithine, norleucine (Nle), and norvaline (Nva).

In the formulae above, n=an integer of 1 to 6; n1=an integer of 1 to 6; n2=an integer of 1 to 6; 0≦n1+n2≦6; m=an integer of 2 to 4; p=an integer of 2 to 4; q=an integer of 1 to 3; and r=an integer of 1 to 3.

Preferred peptides are as follows:

Y-(hydrophobic amino acid);

F-(hydrophobic amino acid);

W-(hydrophobic amino acid);

H-(hydrophobic amino acid);

(hydrophobic amino acid)-Y;

(hydrophobic amino acid)-F;

(hydrophobic amino acid)-W;

(Y/F/W/H)-L-(given amino acid)ₐ;

(Y/F/W/H)-(hydrophobic amino acid)-(Y/F/W/H); and

{(Y/F/W/H)-(hydrophobic amino acid)}ₘ; wherein

the hydrophobic amino acid(s), given amino acid, and (Y/F/W/H) are as defined above.

Cases where the Active Ingredient is a Peptide Analogue

The peptide analogs include the following analogs of the above-mentioned active ingredient peptides: (1) N-terminal modified analogs; (2) C-terminal modified analogs; and (3) analogs of the tyrosine residue, phenylalanine residue, and the like.

(1) The N-terminal amino group of the peptide may be an amino group monosubstituted or disubstituted with a straight- or branched-chain C₁-C₄ alkyl group such as methylamino, dimethylamino, ethylamino, diethylamino, propylamino, dipropylamino, n-butylamino, di-n-butylamino, or the like. Further, the N-terminal amino group or the side-chain amino group (when the peptide contains Lys) of the peptide may be monosubstituted or disubstituted with an aralkyl group such as benzyl, phenethyl, or the like; or modified with an acyl group such as a straight- or branched-chain C₁-C₆ alkanoyl group, for example, formyl, acetyl, propionyl, butyryl, or isobutyryl, a benzoyl group, or the like.

(2) The C-terminal carboxy group of the peptide may form an ester with a C₁-C₆ alkyl group such as methyl, ethyl, n-propyl, isopropyl, n-butyl, isobutyl, t-butyl, pentyl, hexyl, or the like; an ester with an aralkyl group such as benzyl, phenethyl, or the like; an amine monosubstituted or disubstituted with a straight- or branched-chain C₁-C₄ alkyl group such as amino, methylamine, dimethylamine, ethylamine, diethylamine, propylamine, dipropylamine, n-butylamine, di-n-butylamine, or the like; or an amide with ammonia.

(3) Examples of a tyrosine residue analog (I), a phenylalanine residue analog (II), a histidine residue analog (III), and a tryptophan residue analog (IV) include residues represented by the following formulae (I), (II), (III), and (IV), respectively:

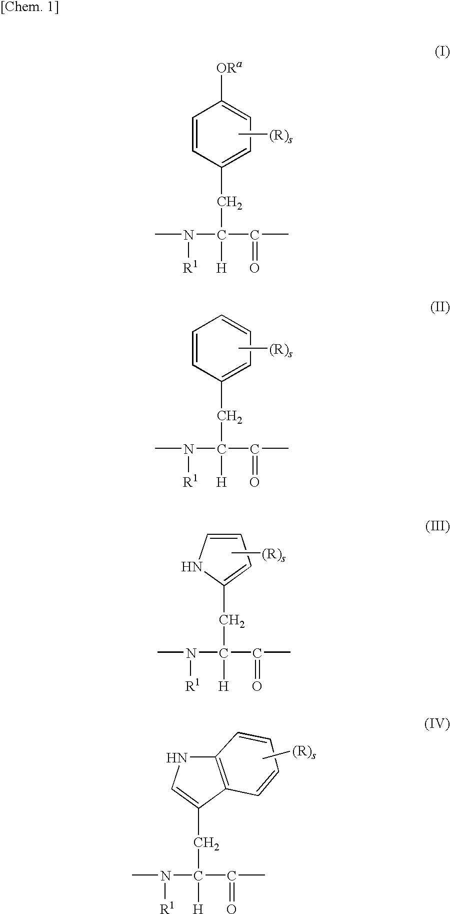

wherein R¹ is a straight- or branched-chain C₁-C₆ alkyl group, aralkyl group, or hydrogen atom; R^{a} is any of a protecting group removable with an acid, such as a hydrogen atom, an alkali metal, an alkaline earth metal, methoxymethyl, 2-tetrahydrofuranyl, or 2-tetrahydropyranyl, methyl, and trifluoromethyl; Rs are each independently a straight- or branched-chain C₁-C₆ alkyl group (for example, methyl, ethyl, n-propyl, isopropyl, n-butyl, isobutyl, t-butyl, pentyl, or hexyl); an aralkyl group (for example, benzyl or hexyl); a straight- or branched-chain C₁-C₆ alkoxy group (for example, methoxy, ethoxy, n-propyloxy, isopropyloxy, n-butoxy, isobutoxy, t-butoxy, pentoxy, or hexyloxy); SH; a straight- or branched-chain C₁-C₆ alkylthio group (for example, methylthio, ethylthio, n-propylthio, isopropylthio, n-butylthio, isobutylthio, t-butylthio, pentylthio, or hexylthio); CN; NO₂; a halogen atom (F, Cl, Br, or I); amino (NH₂); a mono- or di-(C₁-C₆ lower alkyl)amino (for example, methylamino, ethylamino, propylamino, butylamino, dimethylamino, diethylamino, dipropylamino, or dibutylamino); acetamide; acetyl; trifluoromethyl; or hydroxy; and two adjacent Rs or adjacent R and OR^{a} are, when taken together, methylenedioxy or ethylenedioxy.

The active ingredient peptide for use in the drug or foodstuff of the invention (an anxiolytic drug, a sleep-enhancing drug, a drug for treating schizophrenia, or an antidepressant drug) may preferably be YL, FL, WL, HL, YI, FI, YV, LY, LF, LW, IY, IF, (Y/F/W/H)L(Y/F/W), (Y/F/W/H)LQ, L(Y/F/W)L, or (Y/F/W/H)L(Y/F/W)EIAR; and more preferably YL, FL, WL, HL, YI, FI, FV, LY, LF, LW, IY, IF, YLY, YLQ, LYL, or YLYEIAR.

While some of the peptides of the invention and analogs thereof have ACE inhibitory effects, there appears to be no correlation between ACE inhibitory effects and anxiolytic effects; for example, YL having ACE inhibitory effects weaker than those of IY demonstrates stronger anxiolytic effects.

The peptides of the invention can be obtained by hydrolyzing natural proteins or polypeptides, or by chemical synthesis. Examples of proteins and polypeptides to be hydrolyzed include cow milk or human milk casein, α-lactalbumin, β-lactoglobulin, lactoferrin, ovalbumin, bovine and swine myosins, serum albumin, soybean β-conglycinin, glycinin, wheat glutenin, rice glutelin, chloroplast Rubisco, rapeseed napin, and actins known to be widely present in animals and plants (for example, in humans, soybeans, wheat, etc.). The dipeptide sequences of the invention are found in most of the food proteins. These peptides derived from food materials can be incorporated into foodstuffs as they are, or, if necessary, after being subjected to concentration, desalting, purification, or other processing.

The proteolysis may be performed using, for example, a hydrolase such as trypsin, chymotrypsin, papain, pepsin, carboxypeptidase, thermolysin, subtilisin, or the like derived from an animal, plant, or microorganism. The peptide of the invention used as an active ingredient can be obtained using any of these enzymes, by adjusting the pH to a suitable value according to the enzyme, and by allowing the reaction to proceed for about 30 minutes to about 48 hours at about 30 to about 40° C. The peptide of the invention may be purified from the resulting reaction mixture prior to use. When the peptide is obtained by enzymatic degradation of a food material, it may be used as it is, or incorporated into a different food material to prepare a foodstuff or foodstuff composition. The hydrolysis may be performed by reacting a protein in water for 30 minutes to 48 hours at 1 to 100° C. in the presence of a strong acid (for example, hydrochloric acid, nitric acid, or sulfuric acid) or a strong base (for example, an alkali metal hydroxide such as sodium hydroxide, potassium hydroxide, lithium hydroxide, or the like; an alkali metal carbonate such as sodium carbonate, potassium carbonate, or the like; or an alkali metal hydrogencarbonate such as sodium hydrogencarbonate, potassium hydrogencarbonate, or the like), to thereby produce the active ingredient peptide of the invention. The hydrolysis product may be used as it is after pH adjustment, or purified to separate the active ingredient peptide that is to be used.

The peptide of the invention can also be obtained by a peptide synthesis method. Specifically, the condensation can be performed by solution-phase or solid-phase methods generally used in peptide synthesis, such as, for example, a method in which a reactant having a reactive carboxy group and a reactant having a reactive amino group are reacted using an active ester such as HBTU; a method using a coupling agent such as carbodiimide; etc. When the resulting condensation product has a protecting group, the peptide can also be produced by removing the protecting group.

Functional groups that should not be involved in the reaction in this reaction step are protected with protecting groups. Examples of amino-protecting groups include benzyloxycarbonyl (CBZ), t-butyloxycarbonyl (Boc), 9-fluorenylmethyloxycarbonyl (Fmoc), etc. Examples of carboxy-protecting groups include groups capable of forming alkyl esters, benzyl esters, and the like. In the case of a solid-phase method, the C-terminal carboxy group is bonded to a support such as chlorotrityl resin, chloromethyl resin, oxymethyl resin, p-alkoxybenzyl alcohol resin, or the like. The condensation reaction is carried out in the presence of a condensing agent such as carbodiimide, or using an N-protecting amino acid active ester or a peptide active ester.

The protecting group is removed after the completion of the condensation reaction; in the case of a solid-phase method, the bond between the C-terminus of the peptide and the resin is also cleaved. Furthermore, the peptide of the invention is purified according to a general method. Examples of purification methods include ion-exchange chromatography, reverse-phase liquid chromatography, affinity chromatography, and the like. The resulting peptide is analyzed by the Edman degradation technique, using a protein sequencer, GC-MS, or the like that reads an amino acid sequence from the C-terminus.

The peptide of the invention can also be synthesized according to an enzymatic method (see WO2003/010307).

The route of administration of the peptide of the invention is not limited; the peptide can be administered orally, parenterally, or intrarectally. The dose of the peptide will vary depending on the type of compound, the mode of administration, and the age, condition, and the like of the individual administered the peptide; however, the daily dose for an adult is typically 0.01 to 500 mg/kg, preferably 0.05 to 100 mg/kg, and more preferably 0.1 to 30 mg/kg. The peptide (active ingredient) of the invention is typically administered in the form of a pharmaceutical composition in admixture with pharmaceutical carriers. Pharmaceutical carriers that are commonly used in the field of pharmaceutical preparations and that do not react with the peptide of the invention are used.

For example, in the case of peptides having a wide range of effective doses, such as YL, FL, YLYEIAR, and the like, the daily oral dose for an adult may range from about 5 to about 500 mg, and the daily parental dose by injection or the like for an adult may be slightly smaller than the oral dose (for example, from about 1 to about 100 mg).

In the case of peptides having a relatively narrow range of effective doses, such as WL, HL, and the like, the daily oral dose for an adult may range from about 5 to about 100 mg. Within this range, the optimal dose can be determined according to a general method.

The daily dose for an adult of the peptides other than the foregoing can be readily determined based on the doses of the above-mentioned peptides.

The peptide of the invention can be used by itself as a foodstuff or a pharmaceutical, or can be made into, a food preparation or a pharmaceutical preparation, either alone or together with suitable nontoxic carriers for oral administration, diluents, or excipients. Examples of such foodstuff or pharmaceutical preparations include tablets (uncoated tablets, sugar-coated tablets, effervescent tablets, film-coated tablets, chewable tablets, etc.), capsules, troches, powders, subtle granules, granules, solutions, suspensions, emulsions, pastes, creams, injections (including infusions such as amino acid infusions, electrolytes, etc.), and sustained-release preparations such as enteric-coated tablets, capsules, granules, and the like. The amount of the peptide in a foodstuff can be suitably selected, but is typically in the range of from 0.01 to 100 wt %.

Specific examples of pharmaceutical carriers or carriers for oral administration, diluents, excipients, and like substances that can be added to pharmaceuticals or foodstuffs include lactose, glucose, mannite, dextrin, cyclodextrin, starch, saccharose, magnesium aluminometasilicate, synthetic aluminum silicate, sodium carboxymethyl cellulose, hydroxypropyl starch, calcium carboxymethyl cellulose, ion exchange resins, methylcellulose, gelatin, gum arabic, hydroxypropylcellulose, hydroxypropylmethylcellulose, polyvinylpyrrolidone, polyvinyl alcohol, light anhydrous silicic acid, magnesium stearate, talc, tragacanth, bentonite, veegum, titanium oxide, sorbitan fatty acid esters, sodium lauryl sulfate, glycerin, fatty acid glycerol esters, purified lanolin, glycerogelatin, polysorbate, macrogol, vegetable oils, waxes, liquid paraffin, white petrolatum, fluorocarbon, nonionic surfactants, propylene glycol, water, and the like.

Examples of dosage forms include tablets, capsules, granules, powders, syrups, suspensions, suppositories, ointments, creams, gels, patches, inhalants, injections, and the like. These dosage forms are prepared according to general methods. Liquid preparations may be dissolved or suspended in water or other suitable solvents prior to use. Tablets and granules may be coated using known methods. Injections are prepared by dissolving the peptide of the invention in water. As required, injections may also be prepared by dissolving the peptide in physiological saline or glucose solutions, or may additionally contain buffers and preservatives.

These preparations may contain the peptide of the invention in an amount of from 0.01 to 100 wt %, and preferably from 1 to 90 wt %. These preparations may also contain other therapeutically beneficial ingredients.

Solid preparations for oral administration may be prepared by mixing the active ingredient with excipients such as, for example, lactose, starch, crystalline cellulose, calcium lactate, silicic acid anhydride, and the like to form powders; or by further optionally adding thereto binders such as saccharose, hydroxypropylcellulose, and polyvinylpyrrolidone; and disintegrators such as carboxymethyl cellulose and calcium carboxymethyl cellulose; and subjecting the resulting mixtures to wet or dry granulation to form granules. Tablets may be prepared by tableting these powders or granules as they are, or after adding thereto lubricants such as magnesium stearate and talc. These granules or powders can be coated with enteric coatings such as hydroxypropylmethylcellulose phthalate and methacrylate-methyl methacrylate polymer to form enteric-coated preparations; or coated with ethylcellulose, carnauba wax, and hydrogenated oil to form sustained-release preparations. Capsules may be prepared by filling hard gelatin capsules with the powders or granules, or by coating with gelatin films the active ingredient as it is, or after being dissolved in glycerin, polyethylene glycol, sesame oil, olive oil, or the like to form soft gelatin capsules.

Liquid preparations for oral administration may be prepared by dissolving in water the active ingredient together with sweetening agents such as saccharose, sorbitol, and glycerin to form transparent syrups; by further adding thereto essential oils, ethanol, and the like to form elixirs; or by further adding thereto gum arabic, tragacanth, polysorbate 80, sodium carboxymethyl cellulose, and the like to form emulsions or suspensions. These liquid preparations may optionally contain taste-improving agents, coloring agents, preservatives, and the like.

Injections may be prepared by dissolving the active ingredient in distilled water for injection, optionally with pH adjusters such as hydrochloric acid, sodium hydroxide, lactose, lactic acid, sodium, sodium monohydrogen phosphate, and sodium dihydrogen phosphate; and isotonic agents such as sodium chloride and glucose, and filling an ampoule with sterilized and filtered solutions; or by further adding thereto mannitol, dextrin, cyclodextrin, gelatin, and the like, followed by vacuum freeze-drying, to form injections that are reconstituted prior to use. Emulsions for injection can also be prepared by adding to the active ingredient lecithin, polysorbate 80, polyoxyethylene hydrogenated castor oil, and the like, and emulsifying the mixtures in water.

Preparations for rectal or vaginal administration may be prepared by humidifying and dissolving the active ingredient together with suppository bases such as cacao butter, tri-, di-, and monoglycerides of fatty acids, and polyethylene glycol, and by pouring the mixtures into molds, followed by cooling; or by dissolving the active ingredient in polyethylene glycol, soybean oil, and the like, followed by coating the mixtures with gelatin films.

External preparations for the skin may be prepared by adding the active ingredient to white petrolatum, yellow beeswax, liquid paraffin, polyethylene glycol, and the like, and by optionally humidifying and kneading the mixtures to form ointments; or by kneading the active ingredient with adhesives such as rosin and acrylic acid alkyl ester polymers, followed by spreading the mixtures onto nonwoven cloths such as polyalkyls to form tapes.

Specific examples of forms of foodstuffs that can be prepared by adding or blending the peptides of the invention include beverages (for example, coffee, cocoa, juices, soft drinks, mineral drinks, tea beverages, green tea, black tea, oolong tea, milk beverages, lactic acid bacteria beverages, yoghurt beverages, and carbonated beverages), gum, gummy candies, jellies, candies, cookies, crackers, biscuits, ice confectioneries (for example, ice creams, ice candies, sherbets, and shaved ice), retort-pouched foods, jelly-like foods (for example, jellies, agar jelly, and jelly-like beverages), and the like. Foodstuffs that can be prepared by adding or blending the peptides of the invention may take the forms of health foods, functional foods, dietary supplements, foods for specified health uses, foods for the ill/combined foods for the ill (a category of foods for special dietary uses, approved by the Ministry of Health, Labour and Welfare, Japan), and foods for the elderly (a category of foods for special dietary uses, approved by the Ministry of Health, Labour and Welfare, Japan). These foodstuffs may come in the forms of uncoated tablets, film-coated tablets, sugar-coated tablets, granules, powders, tablets, capsules (including both hard and soft gelatin capsules), chewable forms, syrups, drinks, and the like. The preparation of foodstuffs obtained by adding or blending the peptides of the invention can be performed according to known methods.

### EXAMPLES

The present invention will be described in greater detail below with reference to the Examples.

The following Examples, however, do not limit the scope of the invention.

### Elevated Plus-Maze Experiment

The elevated plus-maze (EPM) consisted of two open arms (25×5 cm) and two closed arms (25×5×15 cm), which were joined to a central platform 50 cm high above the floor (see FIG. 1). Because the closed arms were surrounded by fences, a mouse could safely walk in the closed arms irrespective of their elevated position. On the other hand, because the open arms were not surrounded by fences, a mouse walking along the open arms would feel anxious that it might fall down from the elevated position. Therefore, the more time the mouse spent in the open arms, or the greater the number of entries to the open arms, the less anxious the mouse was feeling; thus, the anxiolytic activity was determined based on these indices.

A mouse was placed on a portion of the central platform facing one of the open arms, and the test was started. During a test time of 5 minutes, the cumulative time spent in the open arms (abbr.: time in open arms), the number of visits to the open arms (abbr.: visit to open arms), and the total number of visits to any of the arms (abbr.: total visits) were recorded. The percentage of the time spent in the open arms and the percentage of the number of visits to the open arms were calculated as indices of anxiety.

### Open-Field Test

The open-field used in this experiment was a cylindrical gray apparatus with a diameter of 60 cm and a height of 50 cm, which is divided into 25 sections with black lines. Mice exhibit exploratory behavior when they are placed in a new environment; usually, however, they tend to avoid exploring the center of the apparatus. However, once a mouse is administered an anxiolytic drug, the time spent in the circle at the center and the number of entries thereto increase. Thirty minutes after administration of the peptide, the mouse was placed in the center of the apparatus, and its movement was observed for 5 minutes.

### Statistical Analysis

The data obtained from the elevated plus-maze test were represented as the means±SEM. The data were analyzed by 1-way or 2-way ANOVA, followed by the Fisher test for multiple comparisons.

### Examples 1 to 16 and Comparative Examples 1 to 3

### Anxiolytic Effects

### Experiments and Results

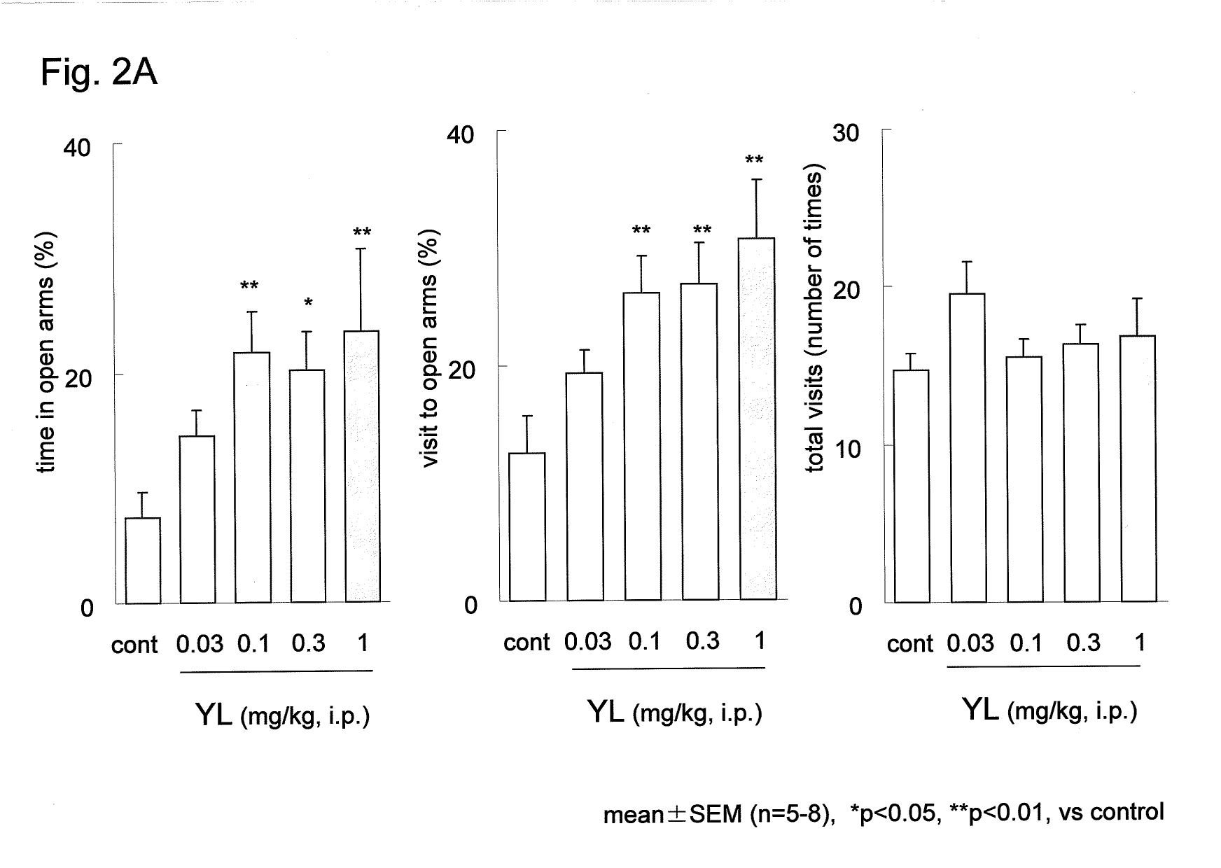

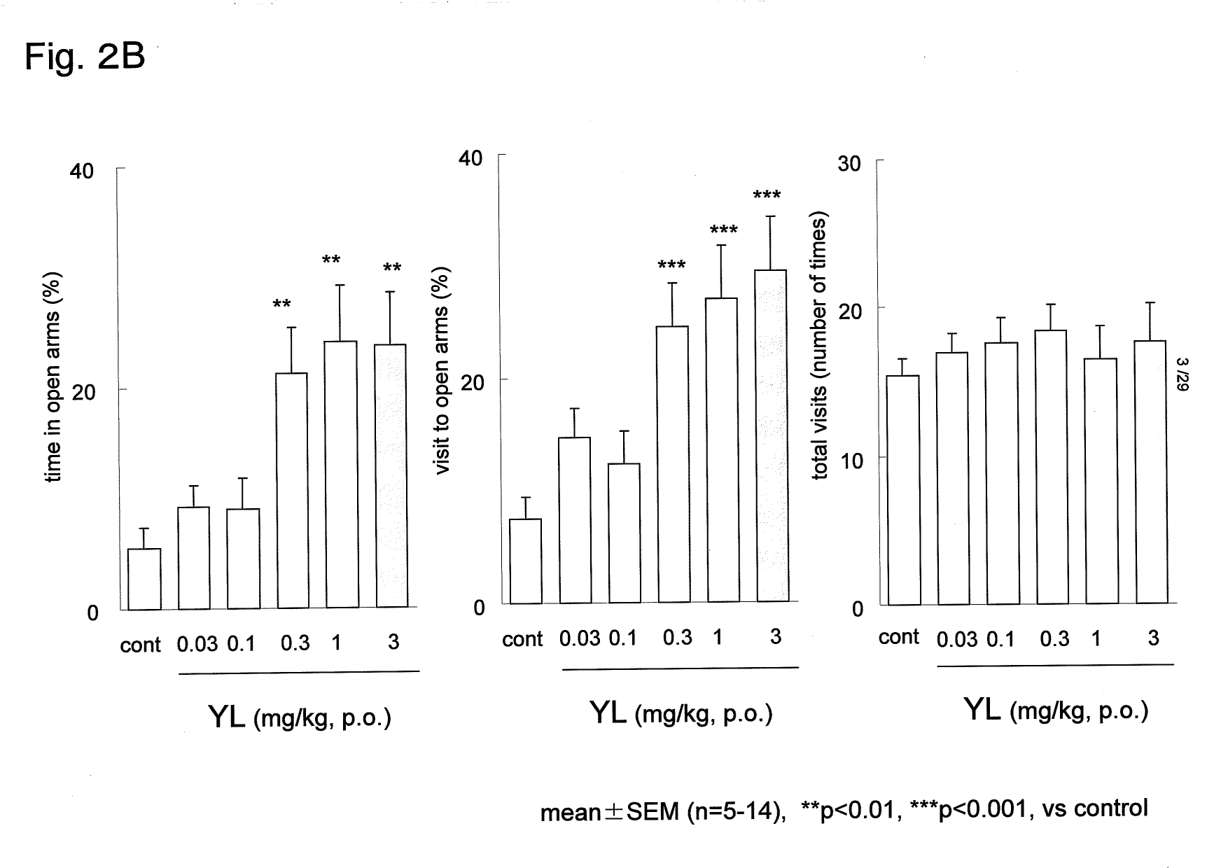

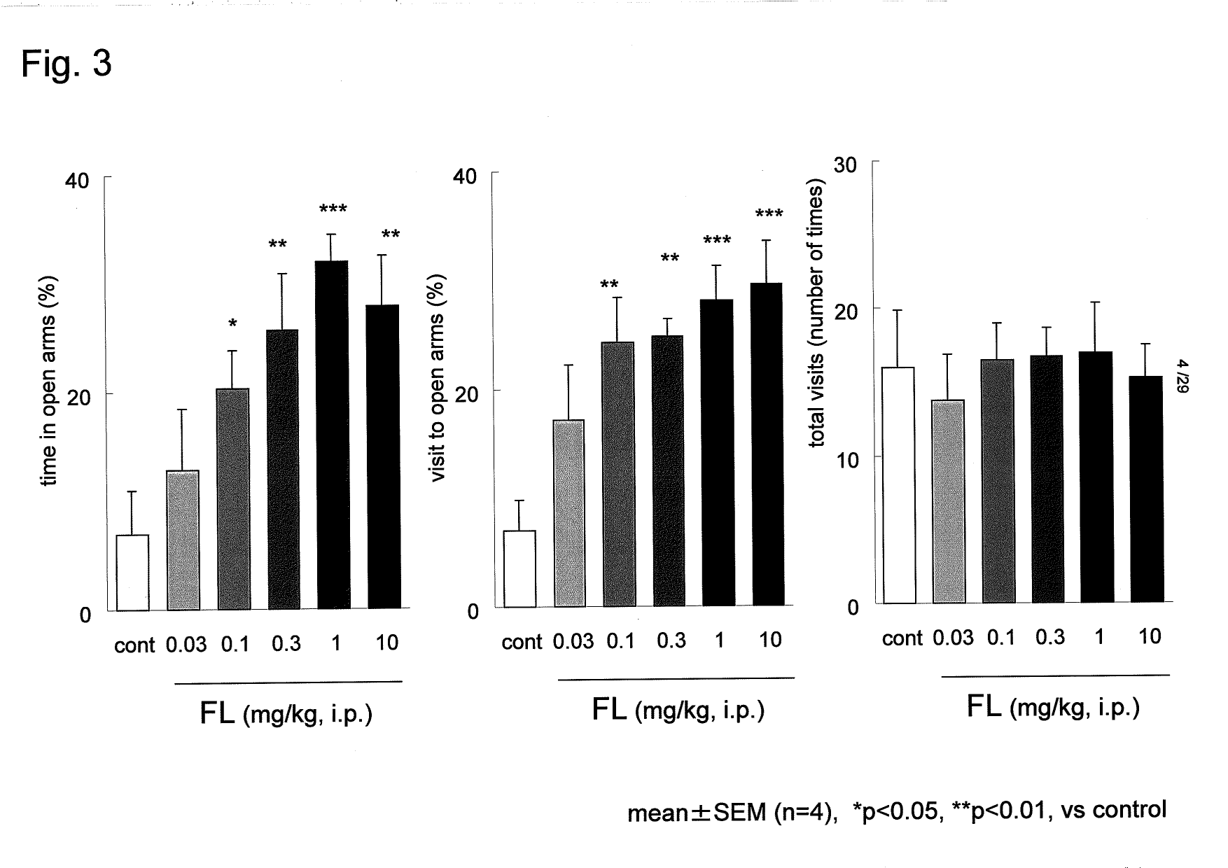

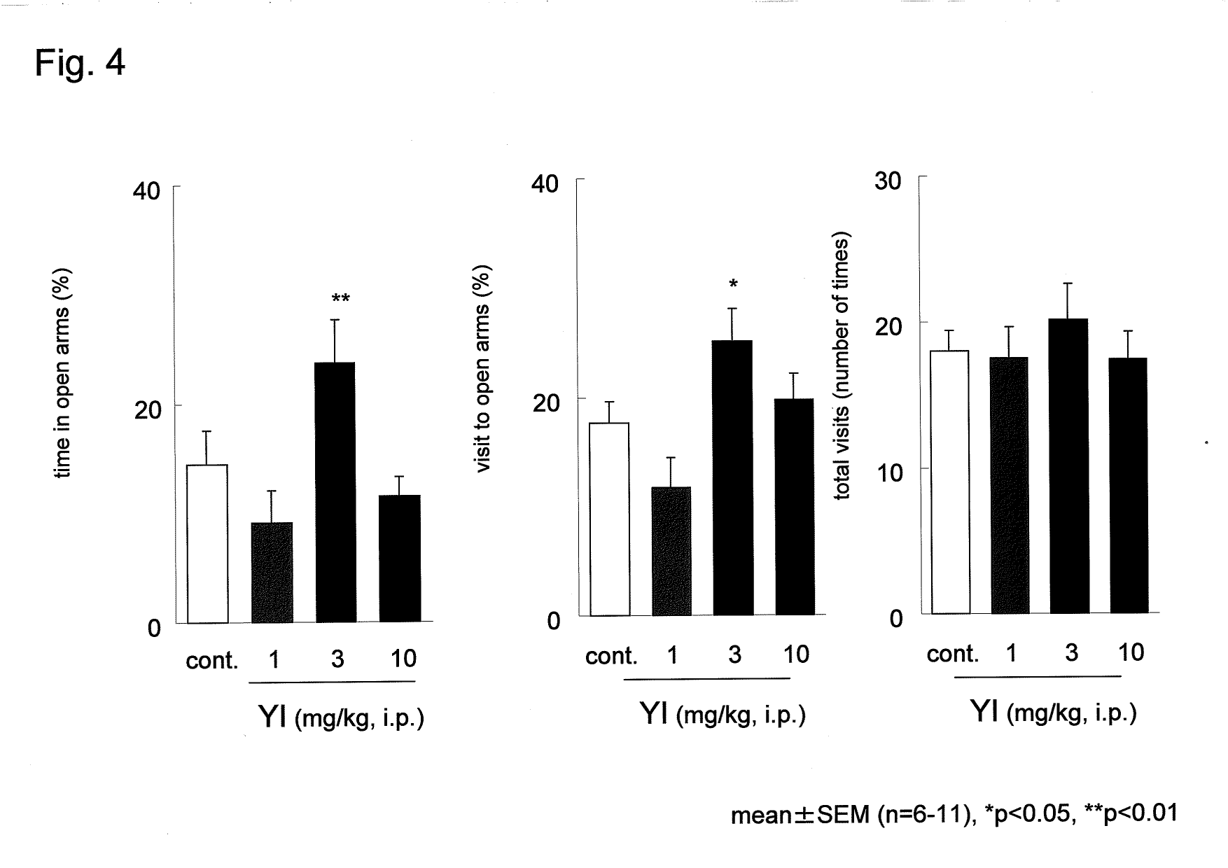

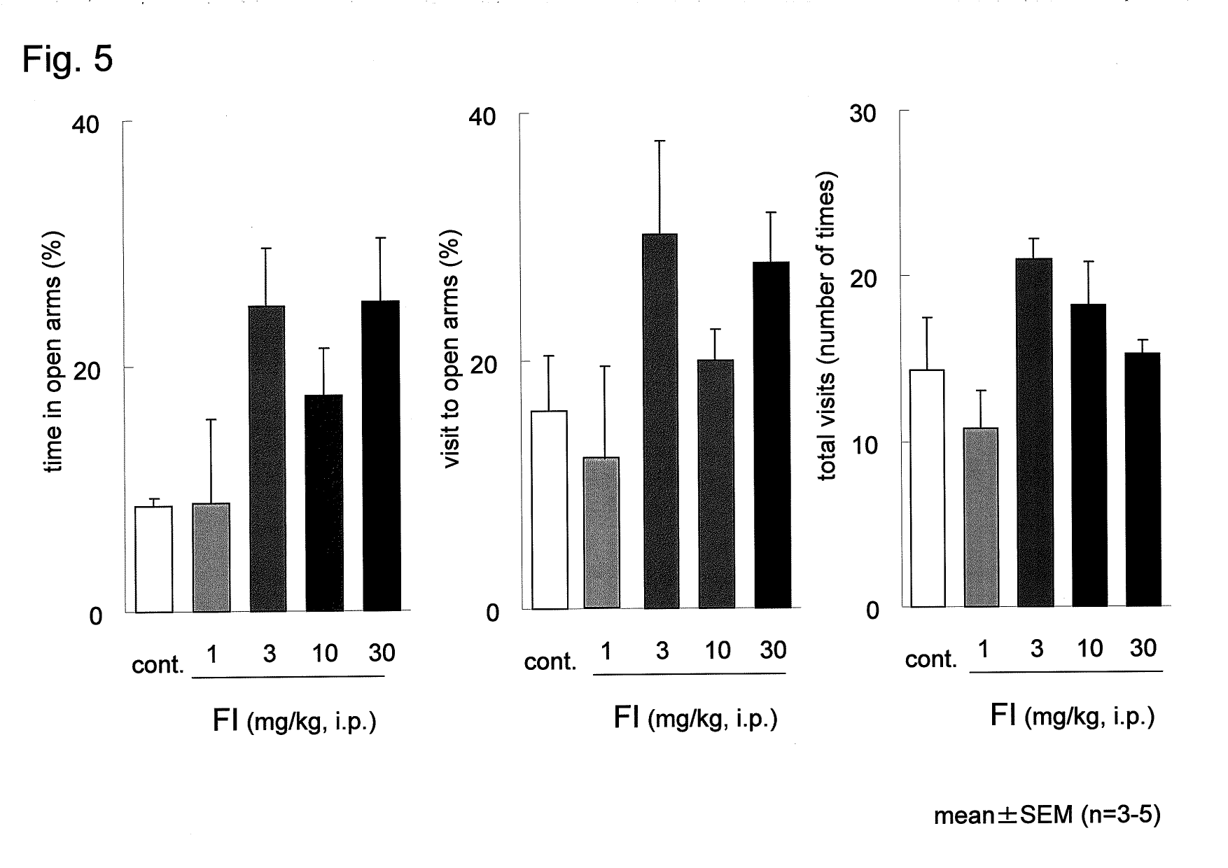

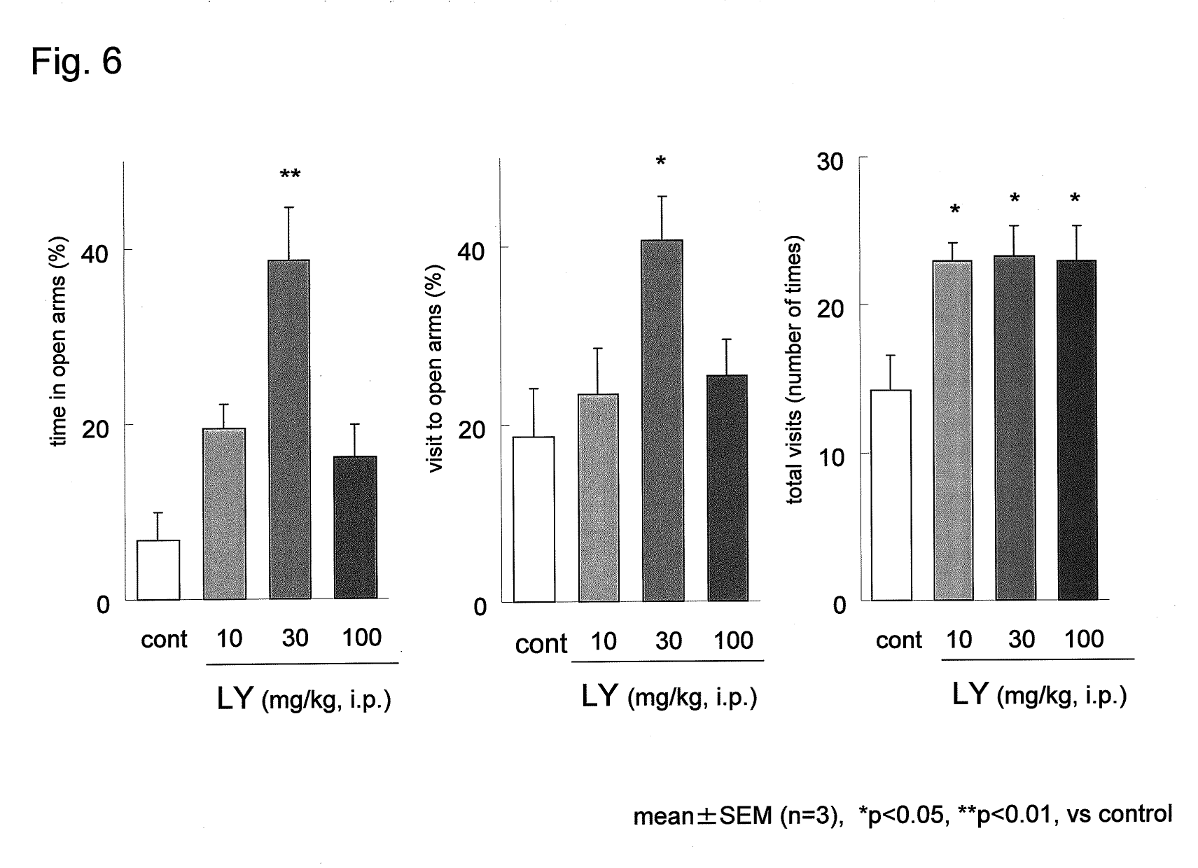

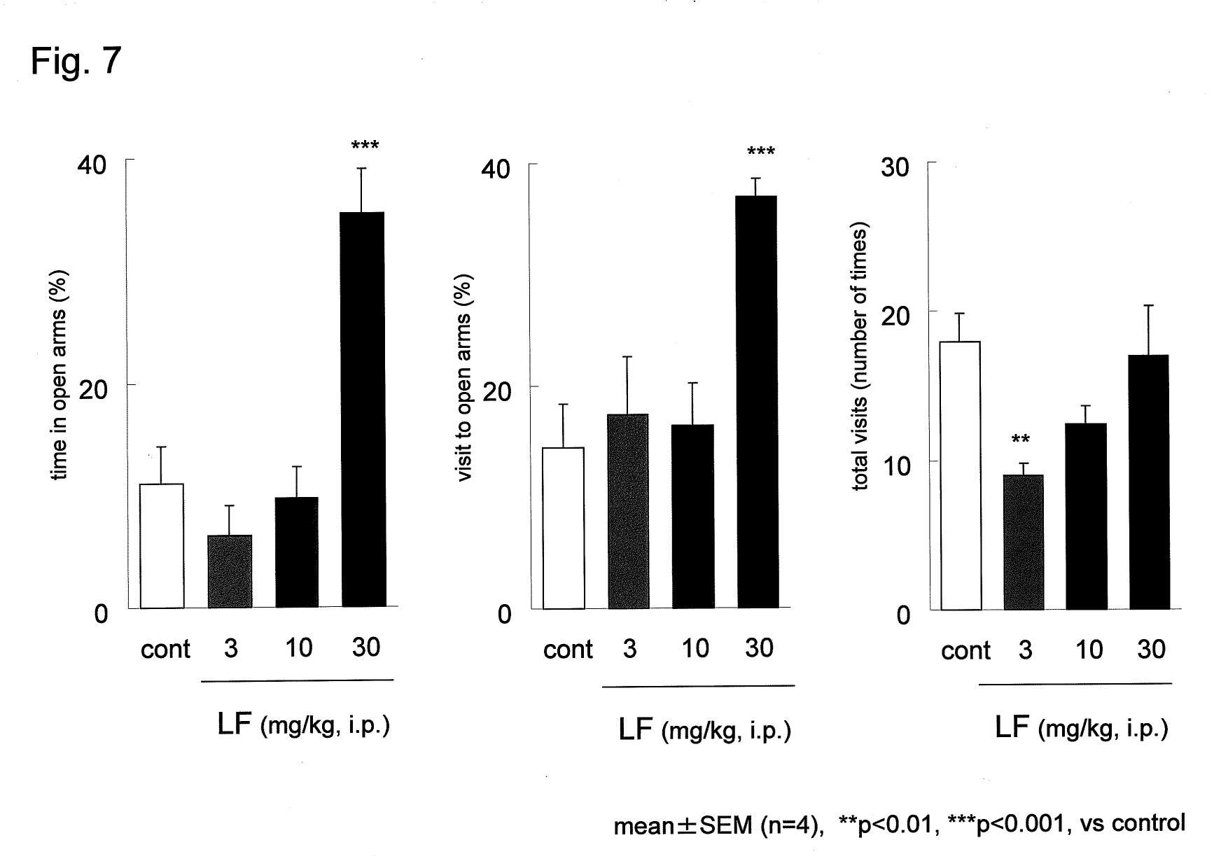

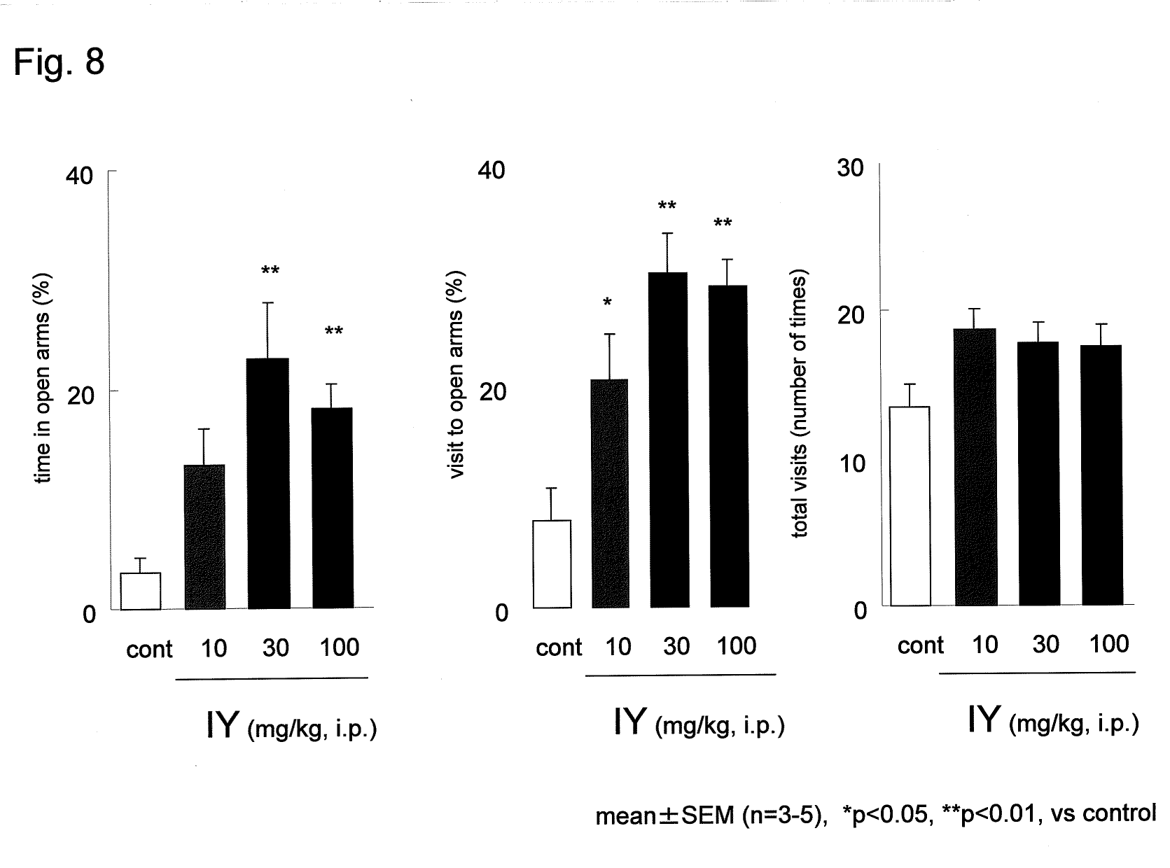

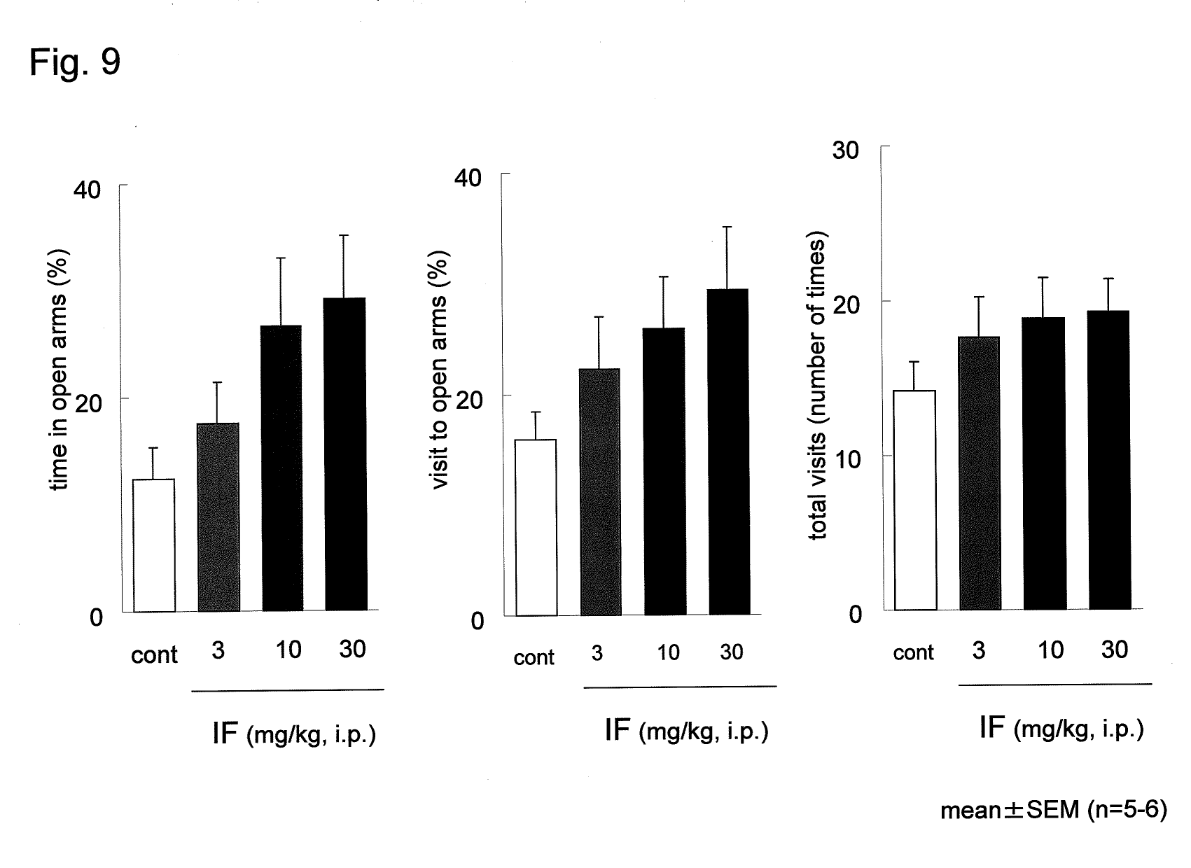

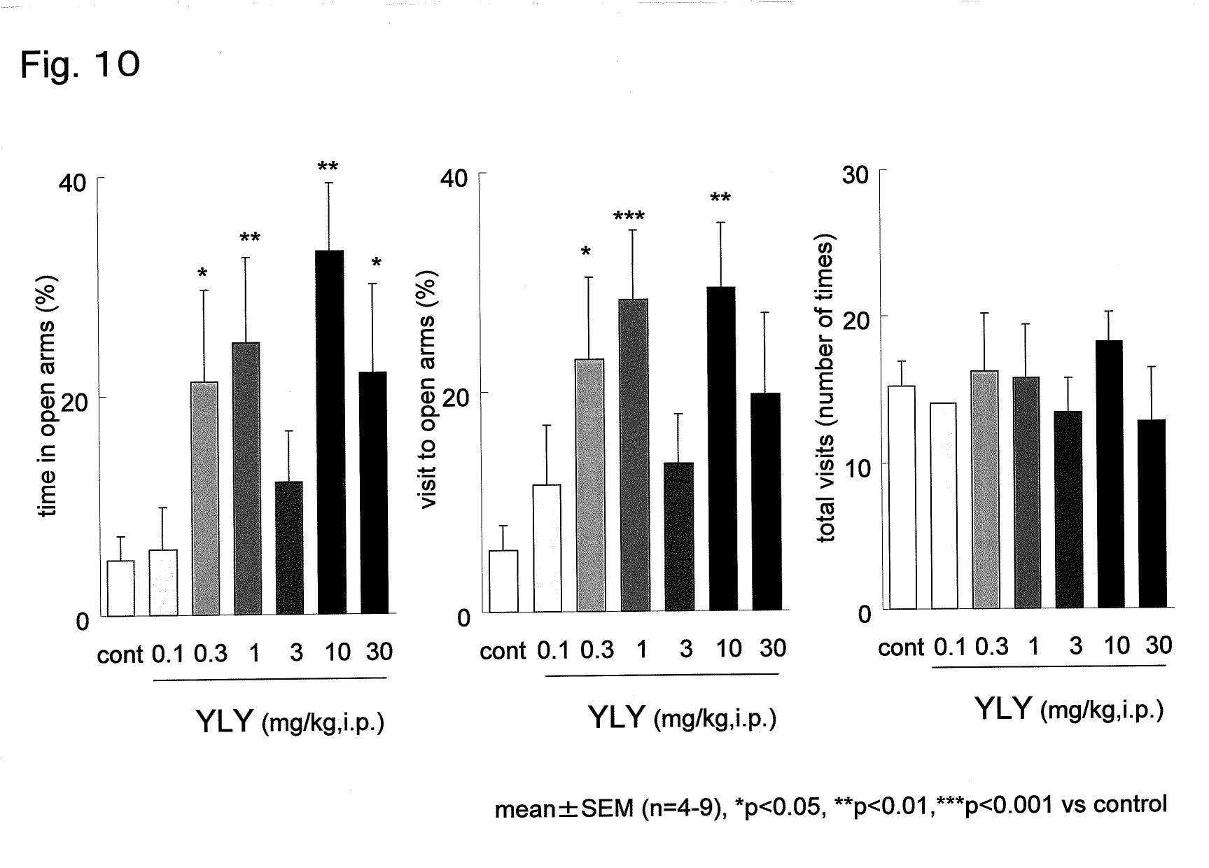

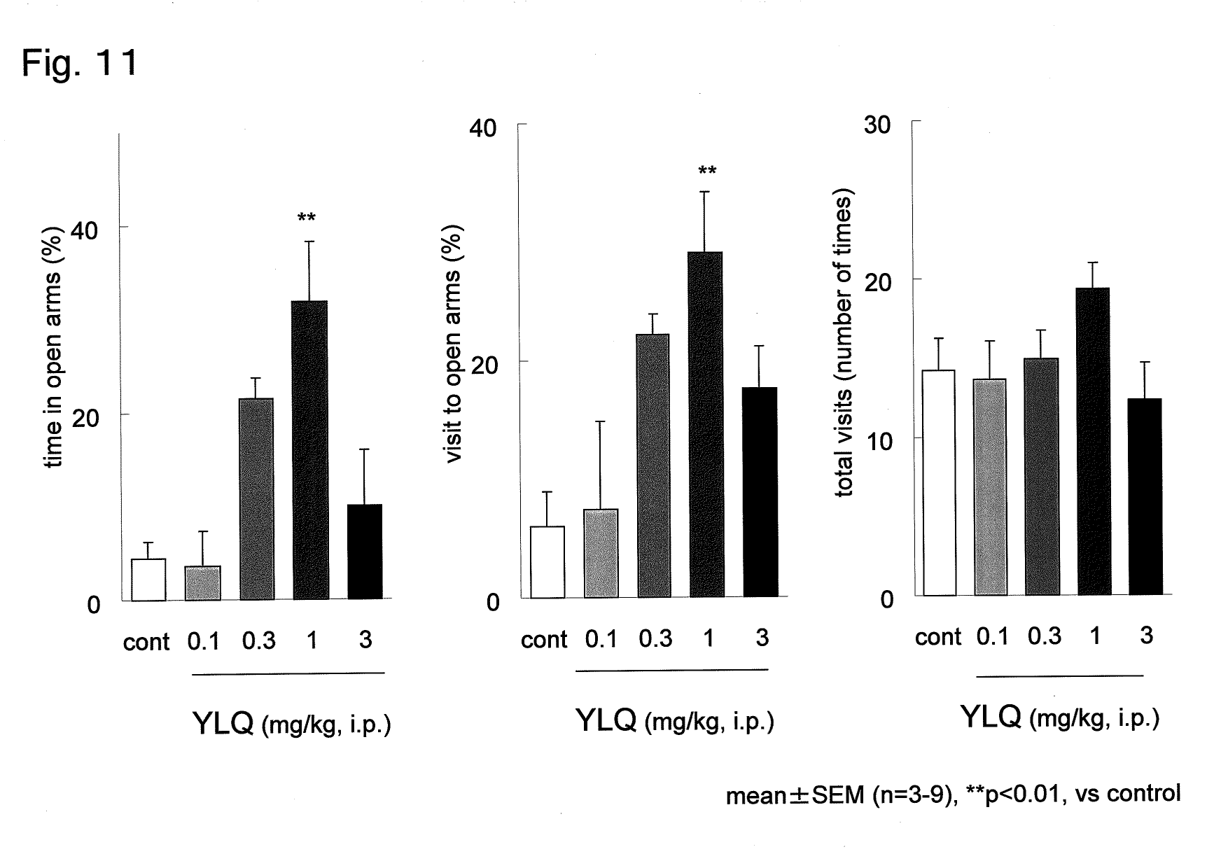

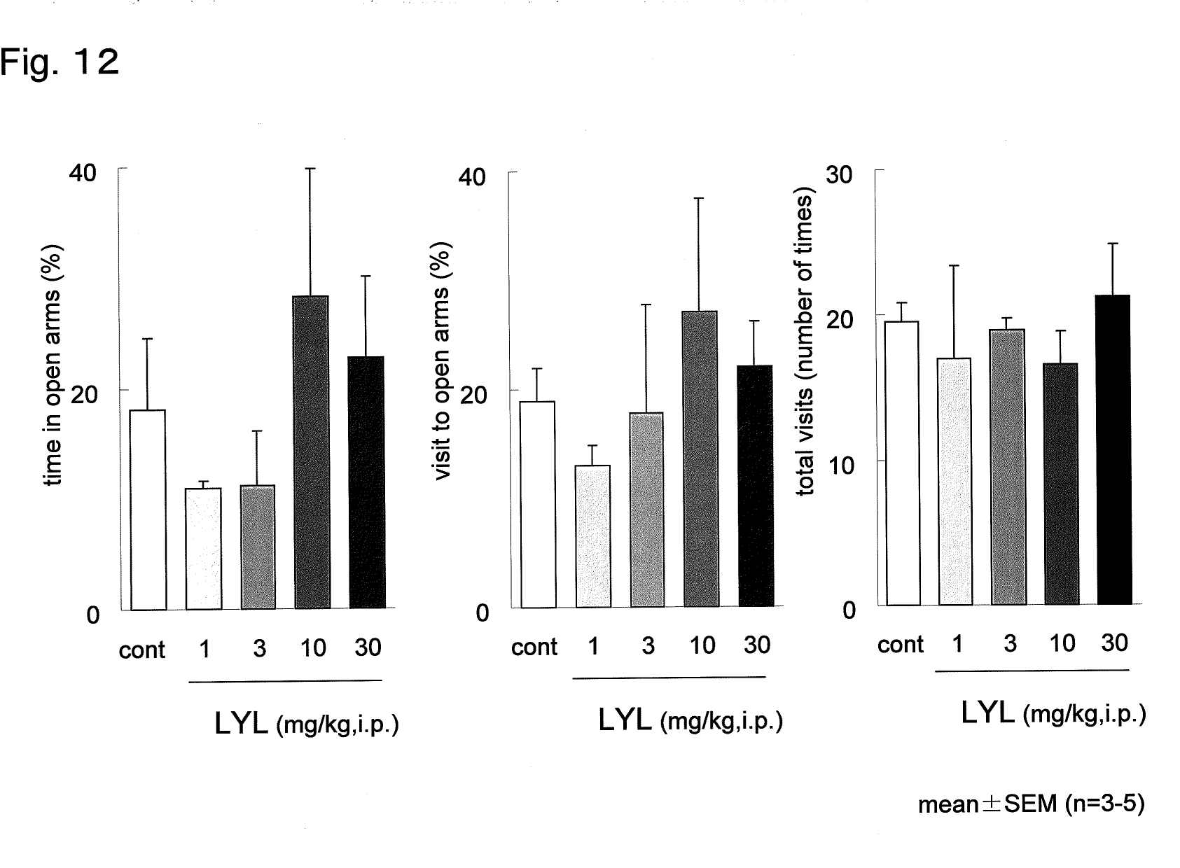

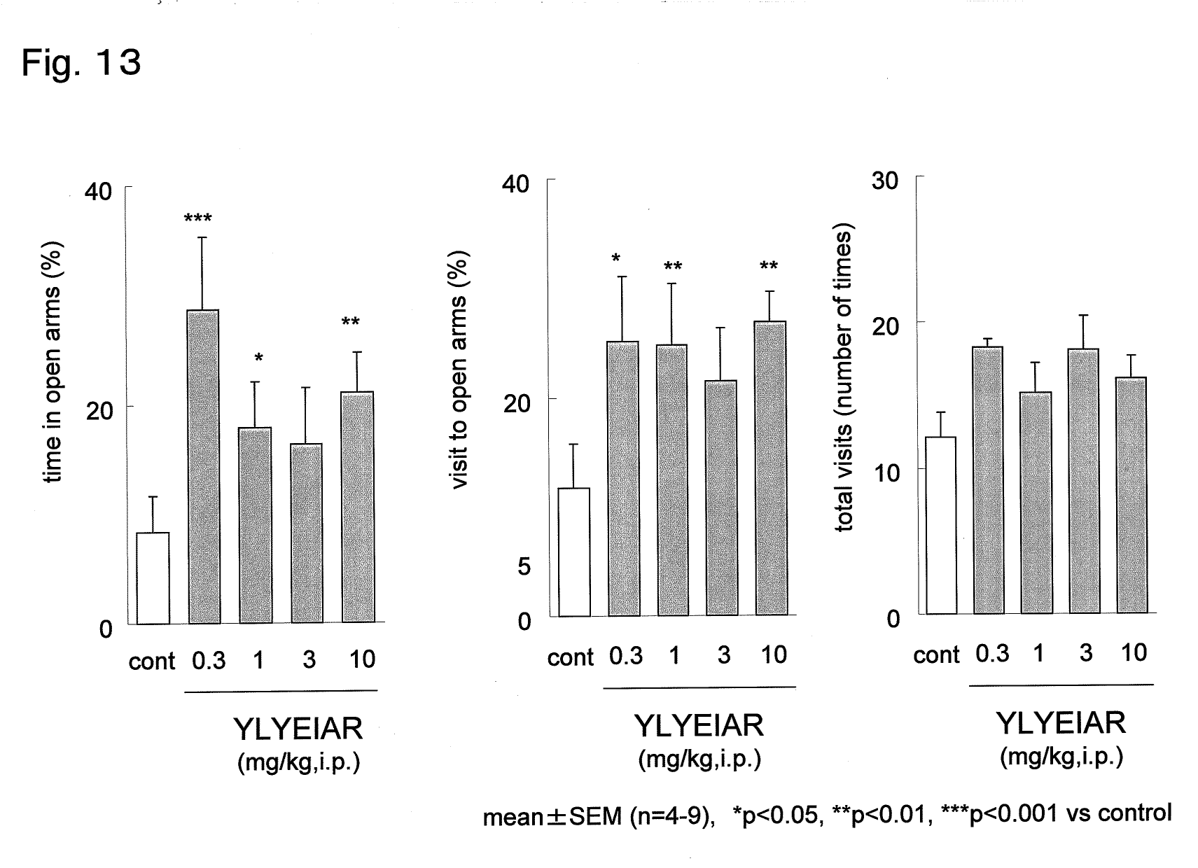

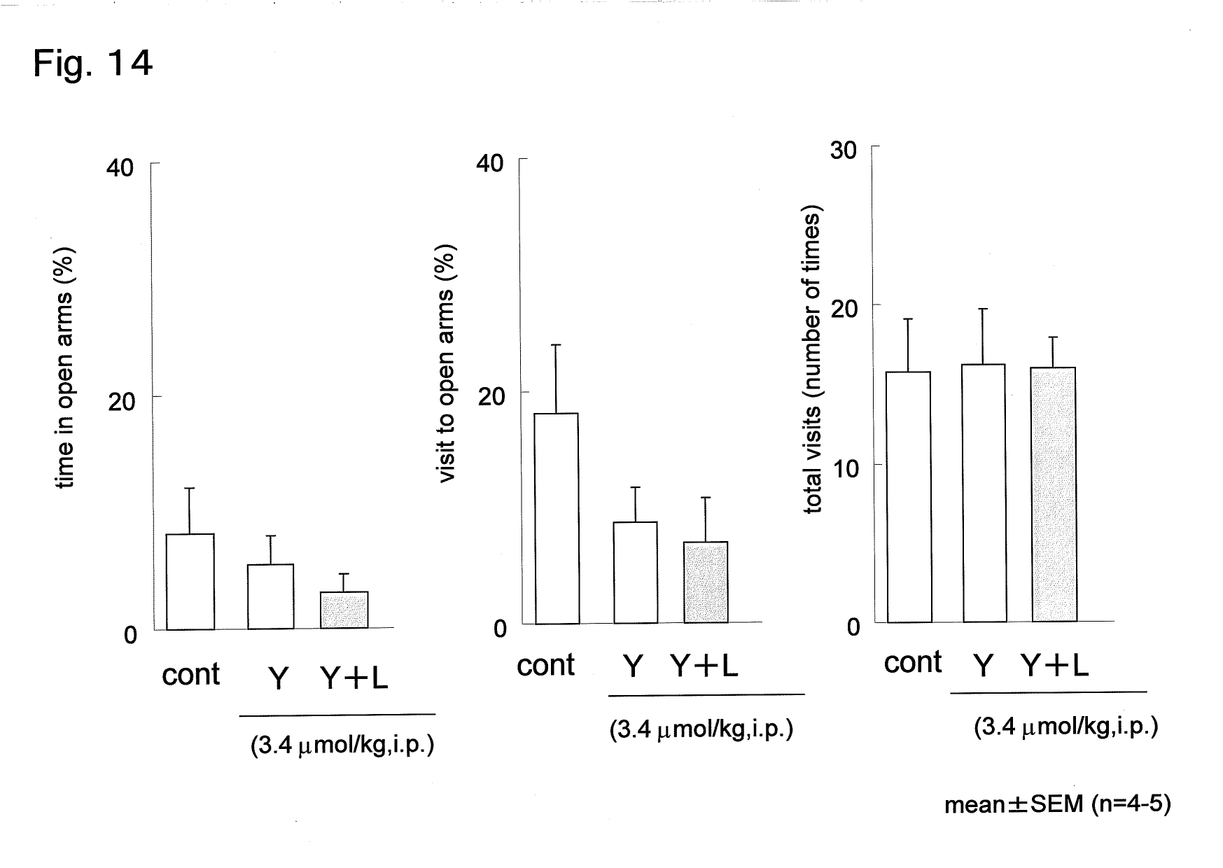

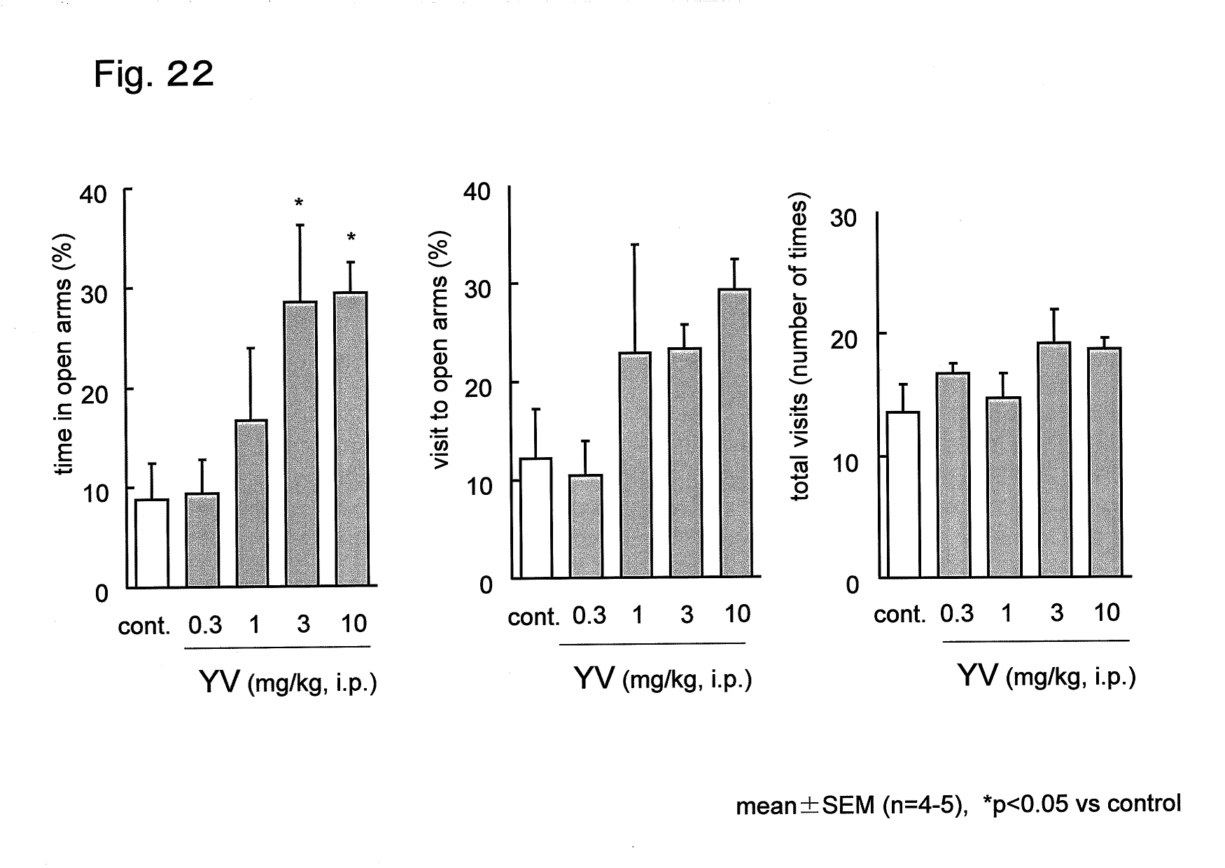

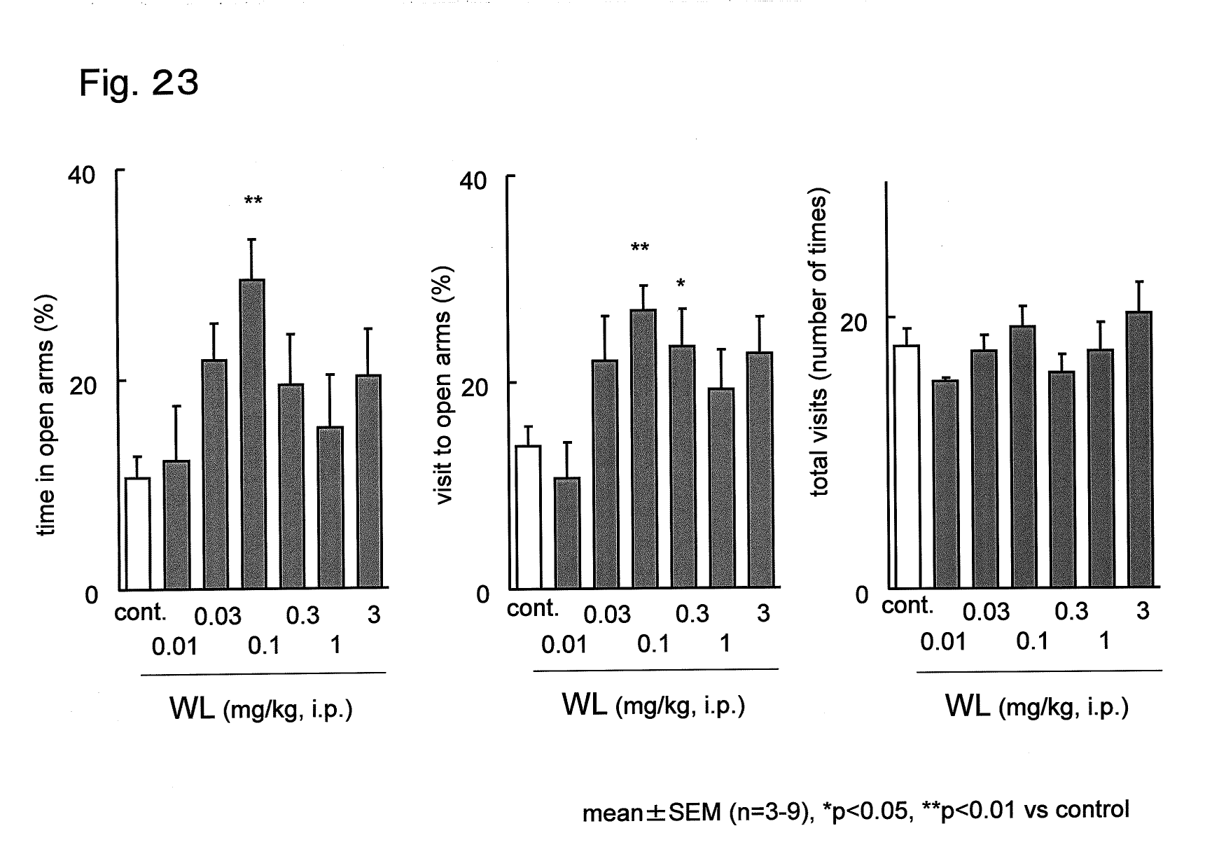

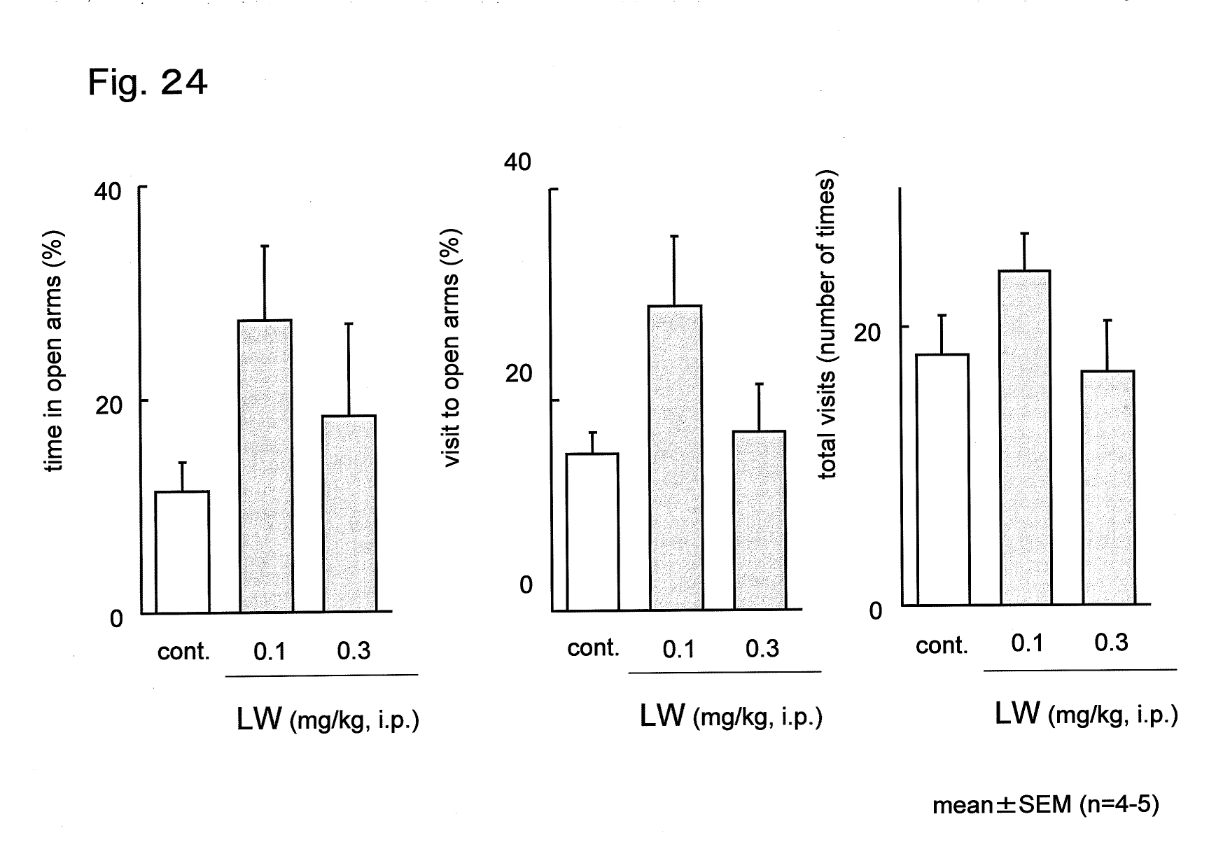

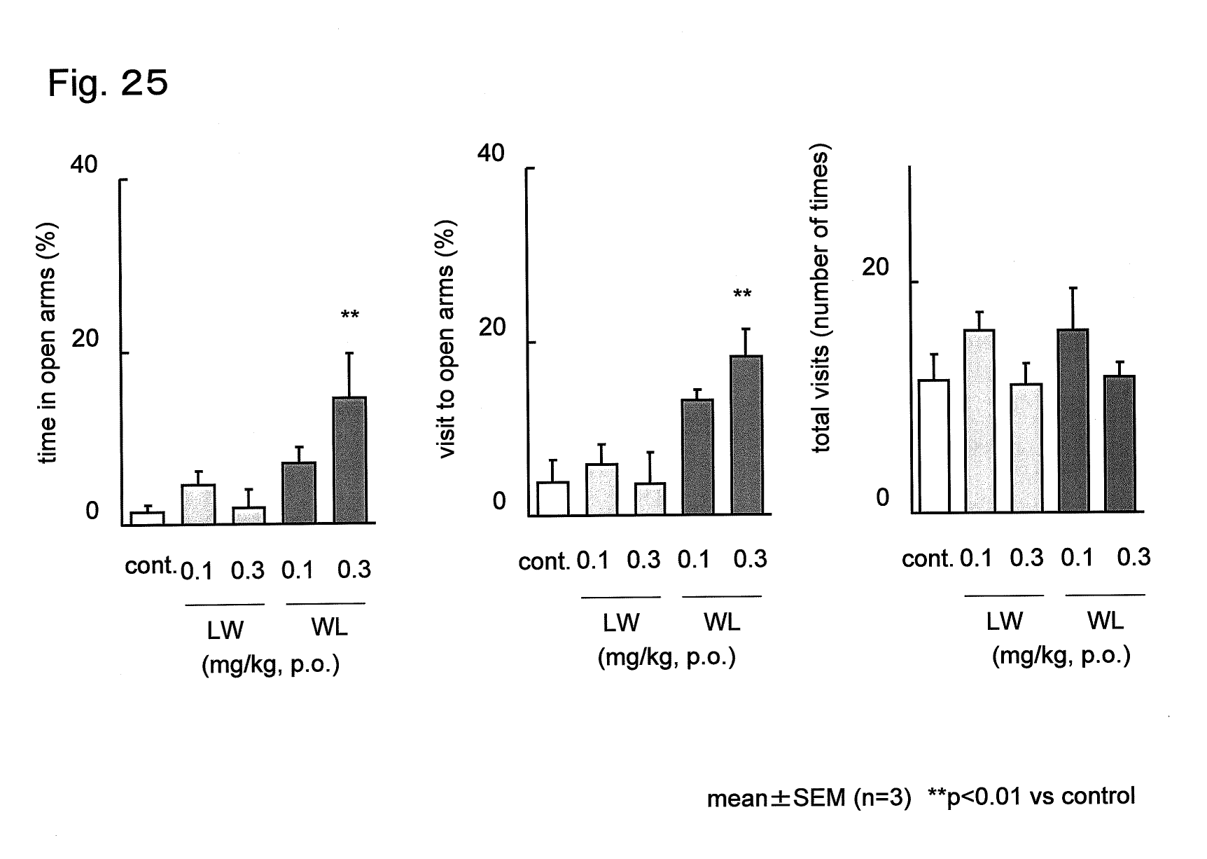

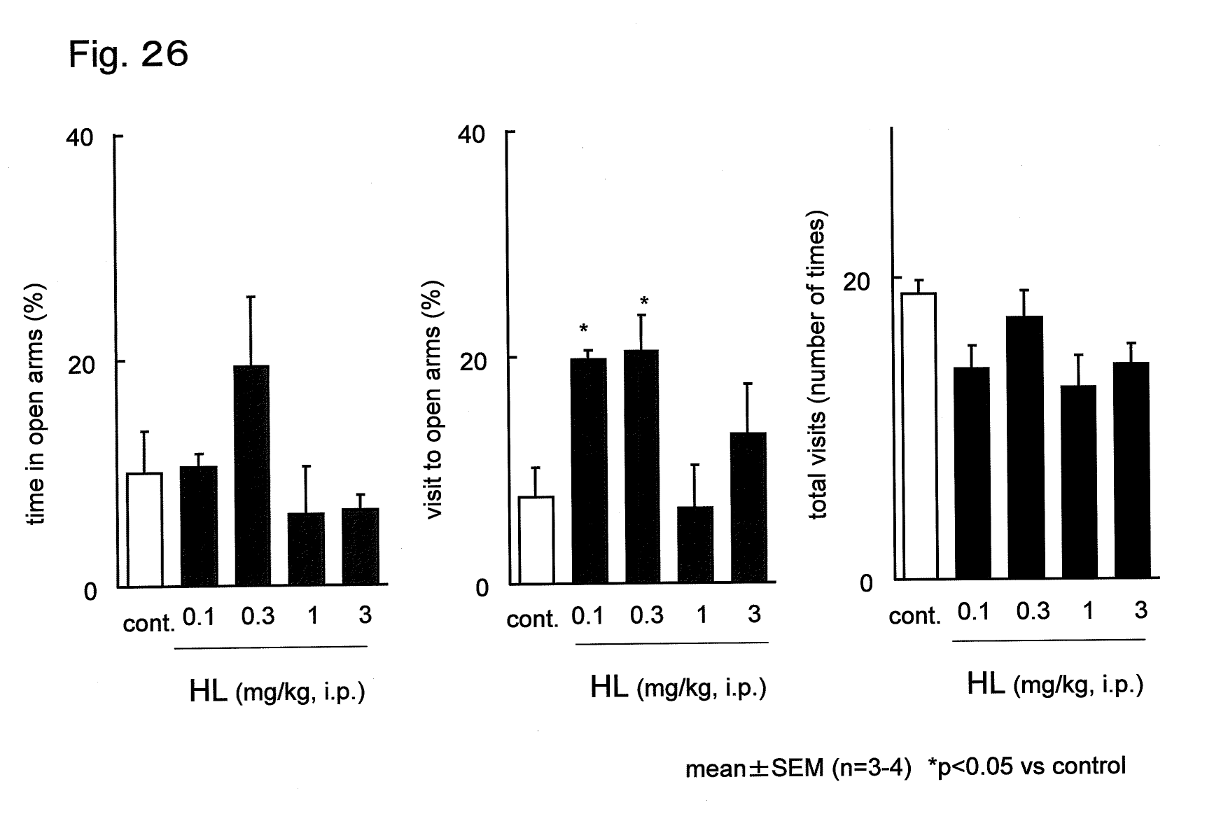

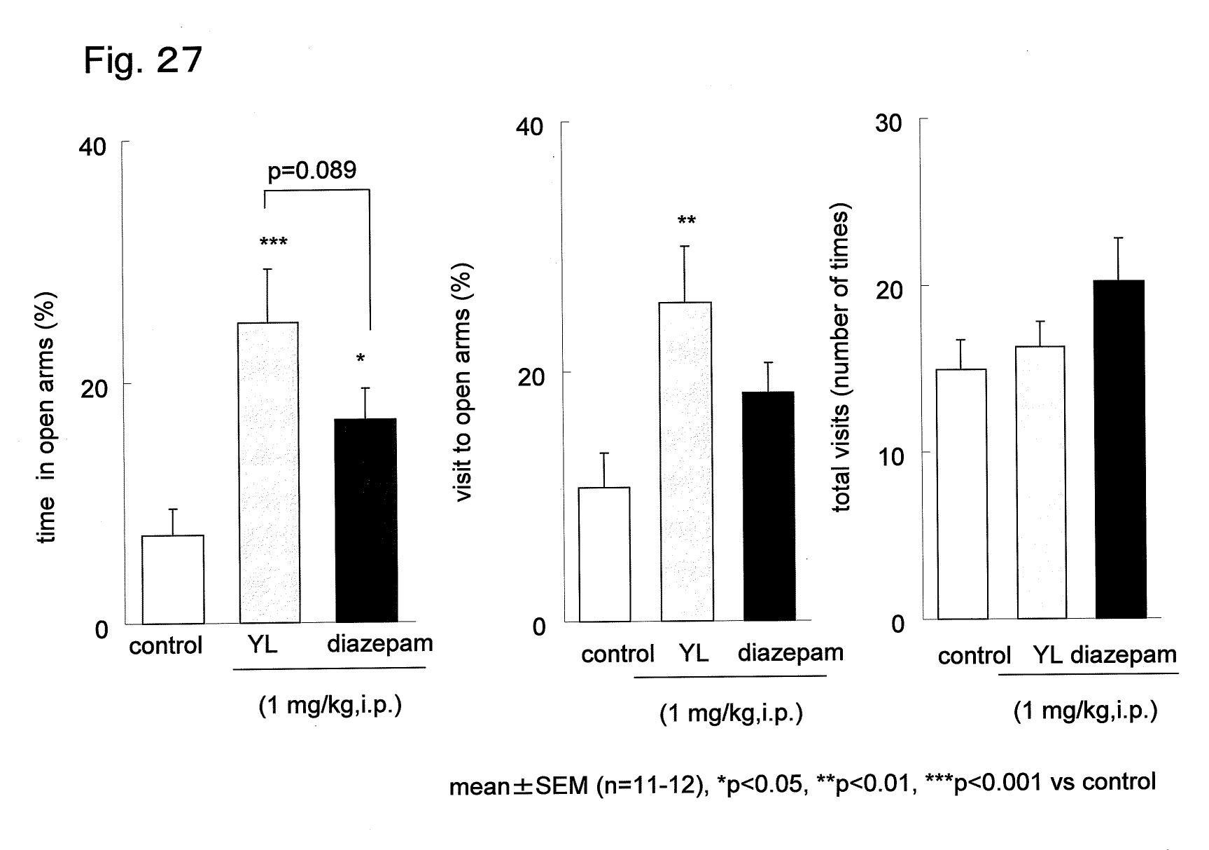

Each of YL (Example 1 and Comparative Example 3), FL (Example 2), YI (Example 3), FI (Example 4), LY (Example 5), LF (Example 6), IY (Example 7), IF (Example 8), YLY (Example 9), YLQ (Example 10), LYL (Example 11), YLYEIAR (Example 12), YV (Example 13), WL (Example 14), LW (Example 15), HL (Example 16), Y (Comparative Example 1), and Y, L (Comparative Example 2) dissolved in physiological saline was administered intraperitoneally (i.p.) or orally (p.o.) to mice (n=3 to 14) in the amounts shown in each figure before the mice were placed in the elevated plus-maze. The percentage of the time spent in the open arms, the percentage of the number of visits to the open arms, and the total number of visits to the arms (total visits) were compared among groups administered each peptide or amino acid, and a control group (0 mg/kg). YL was investigated for its anxiolytic effects by the open-field test. The results are shown in FIGS. 2 to 14, FIGS. 22 to 27, and Table 3. As shown in FIGS. 2 to 14, FIGS. 22 to 27, and Table 3, the peptides of the invention significantly lengthened or showed a significant tendency to increase the percentage of visits to the arms, and the percentage of the time spent in the arms. Furthermore, YL demonstrated anxiolytic effects at least equivalent to those of diazepam in the elevated plus-maze experiment, and also demonstrated its efficacy in the open-field test. In contrast, the two amino acids Y and L were found to have no anxiolytic effects.

Table 1 shows the summarized results.

TABLE 1
Relationship between the structures of the low-molecular-weight
peptides and the anxiolytic effects of the peptides

| Peptide or | Minimum |
|---|---|
| Amino Acid | Effective Dose |
| Administered | (mg/kg, i.p.) |
| YL | 0.1 |
| FL | 0.1 |
| WL | 0.1 |
| HL | 0.1 |
| YI | 3 |
| FI | (3) |
| YV | 3 |
| LY | 30 |
| LF | 30 |
| LW | (0.1) |
| IY | 10 |
| IF | (30) |
| YLY | 0.3 |
| YLQ | 1 |
| LYL | (10) |
| YLYEIAR | 0.3 |
| Y | — |
| L | — |

( ): showed tendency
—: no anxiolytic effects

### Test Example 1

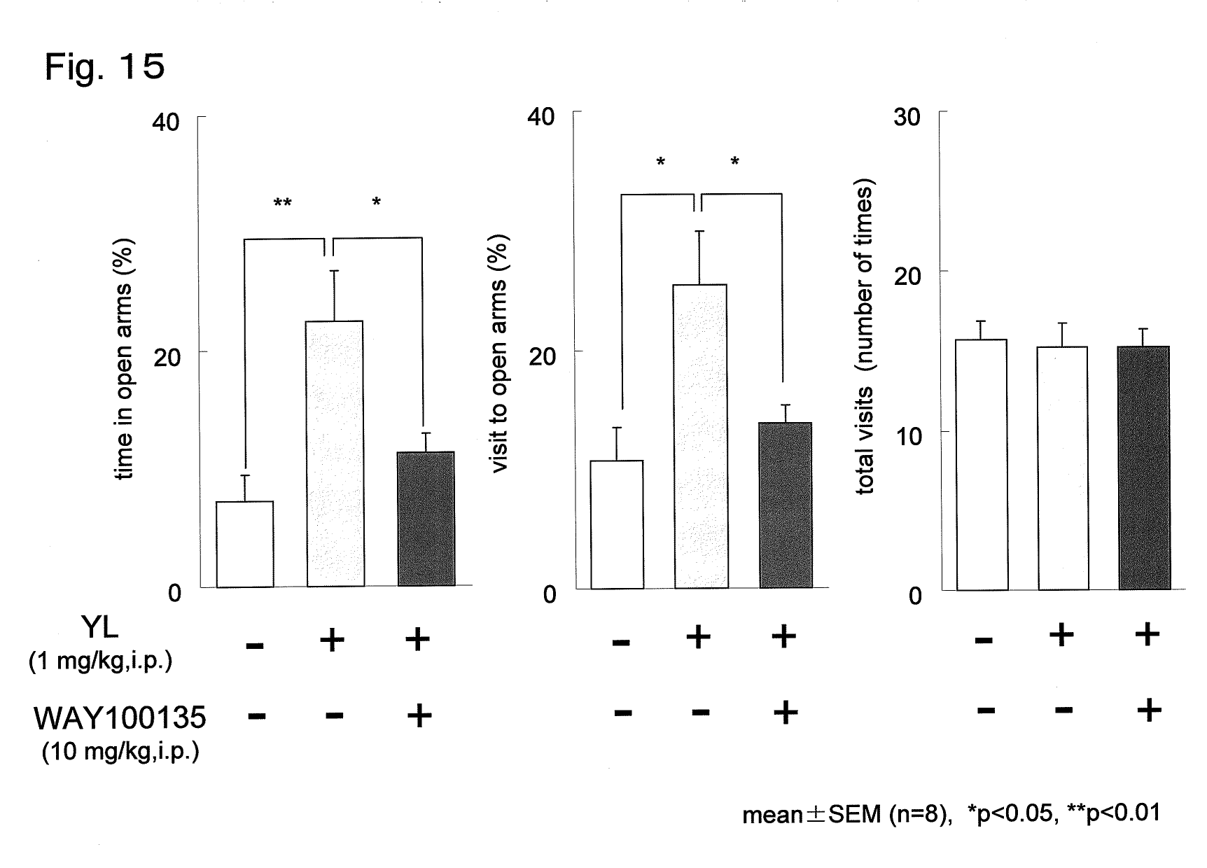

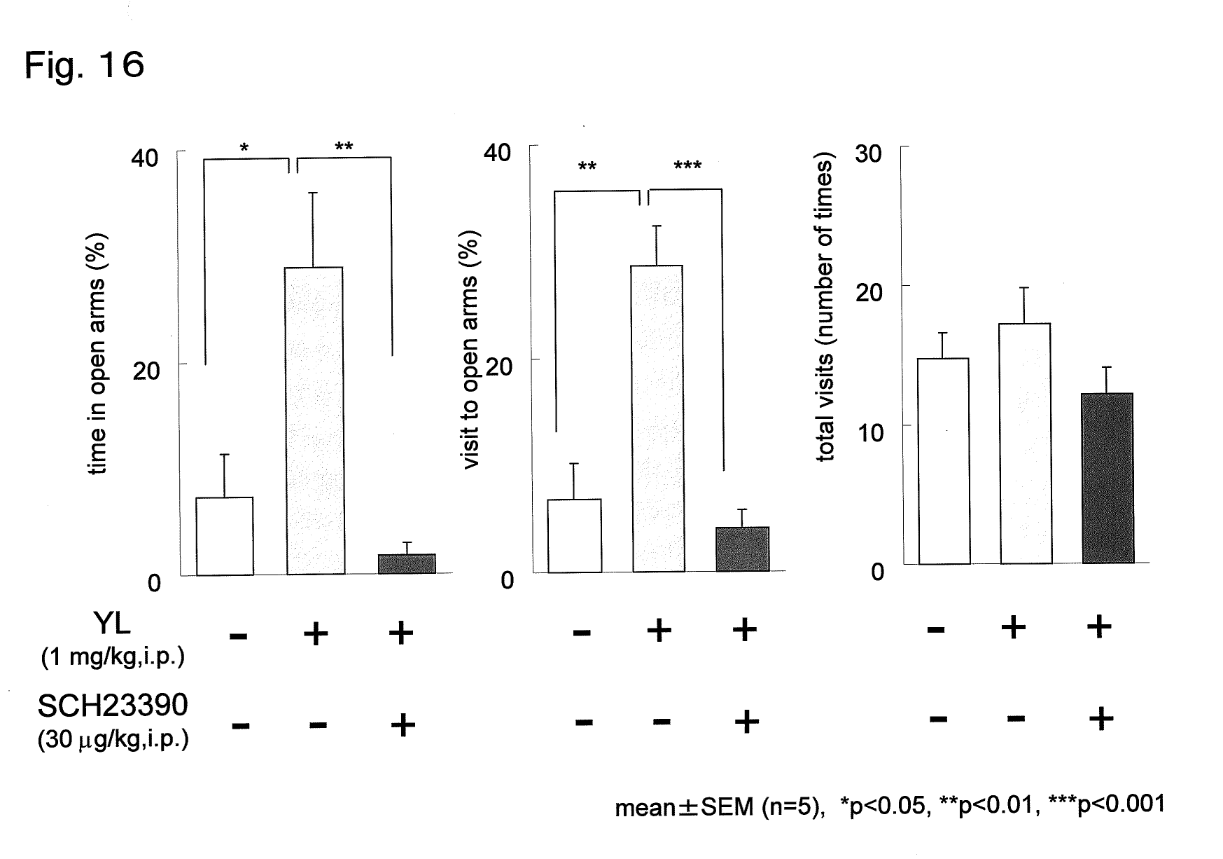

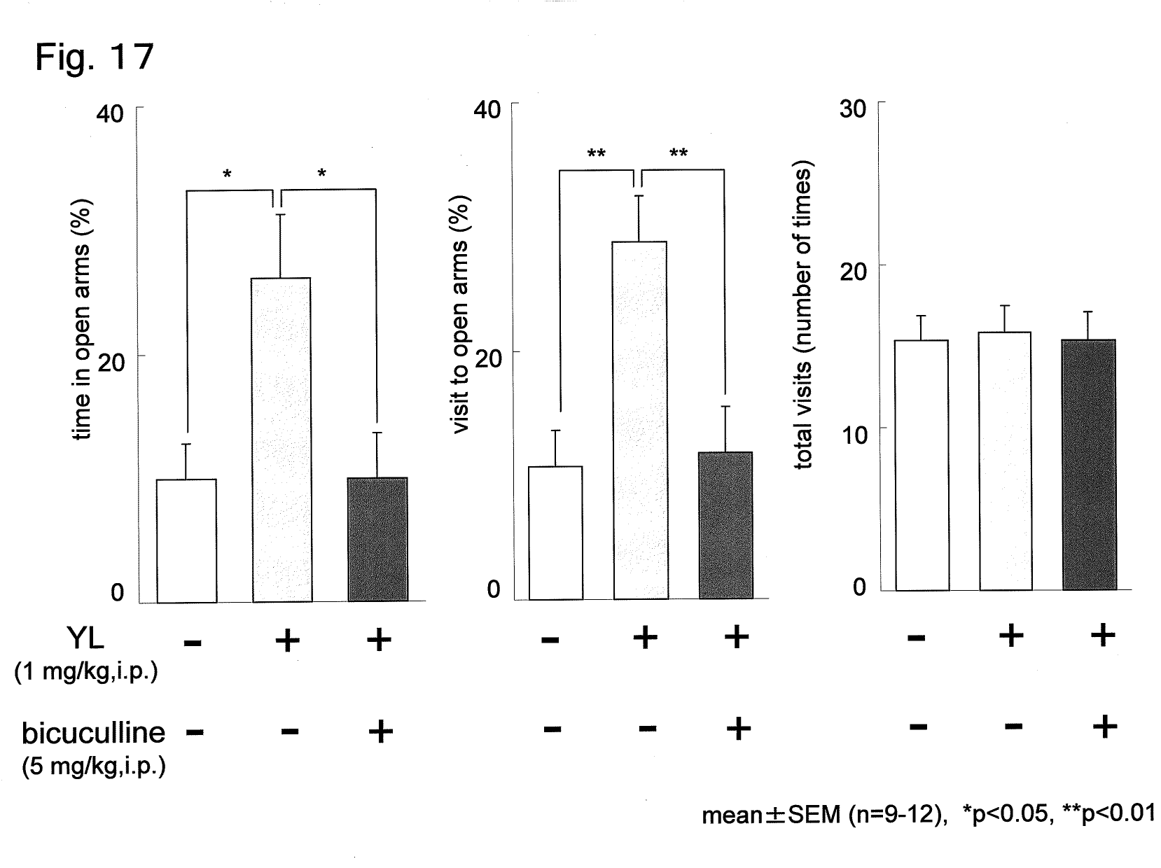

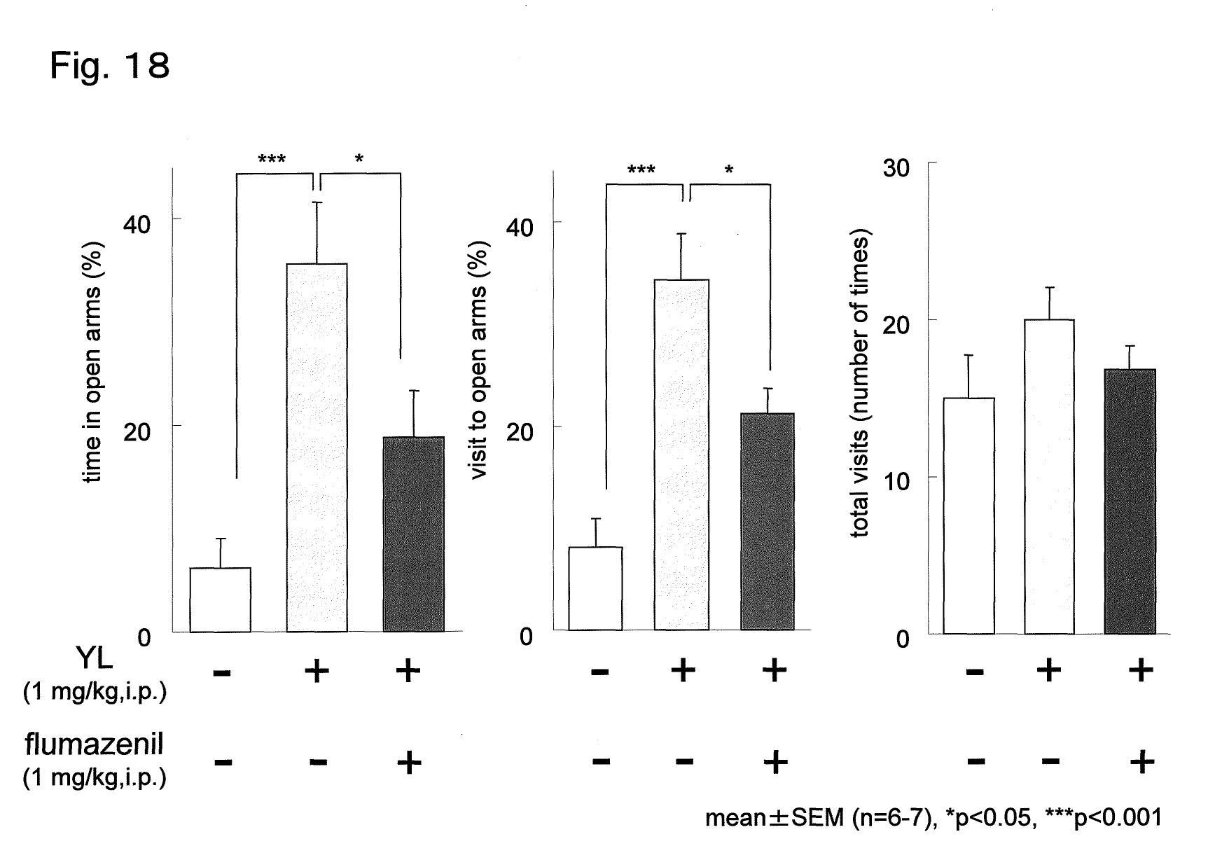

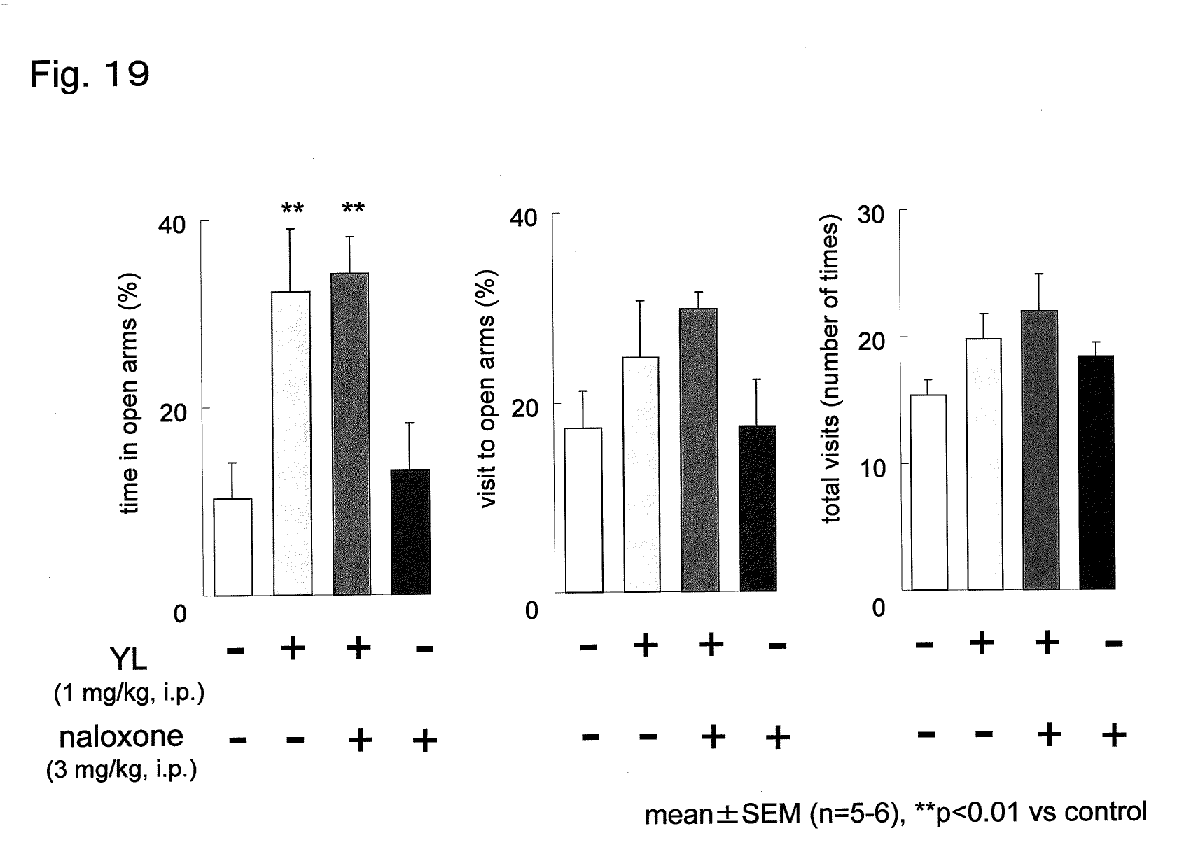

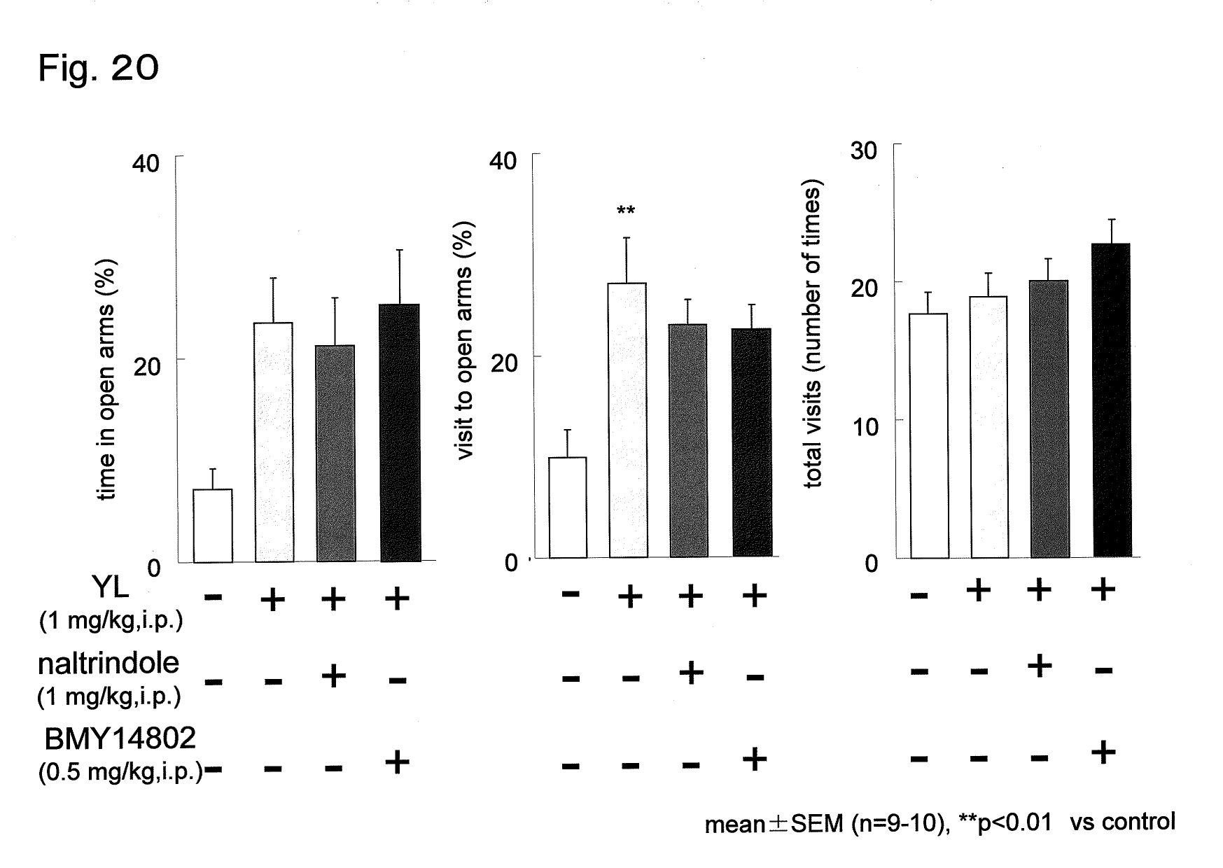

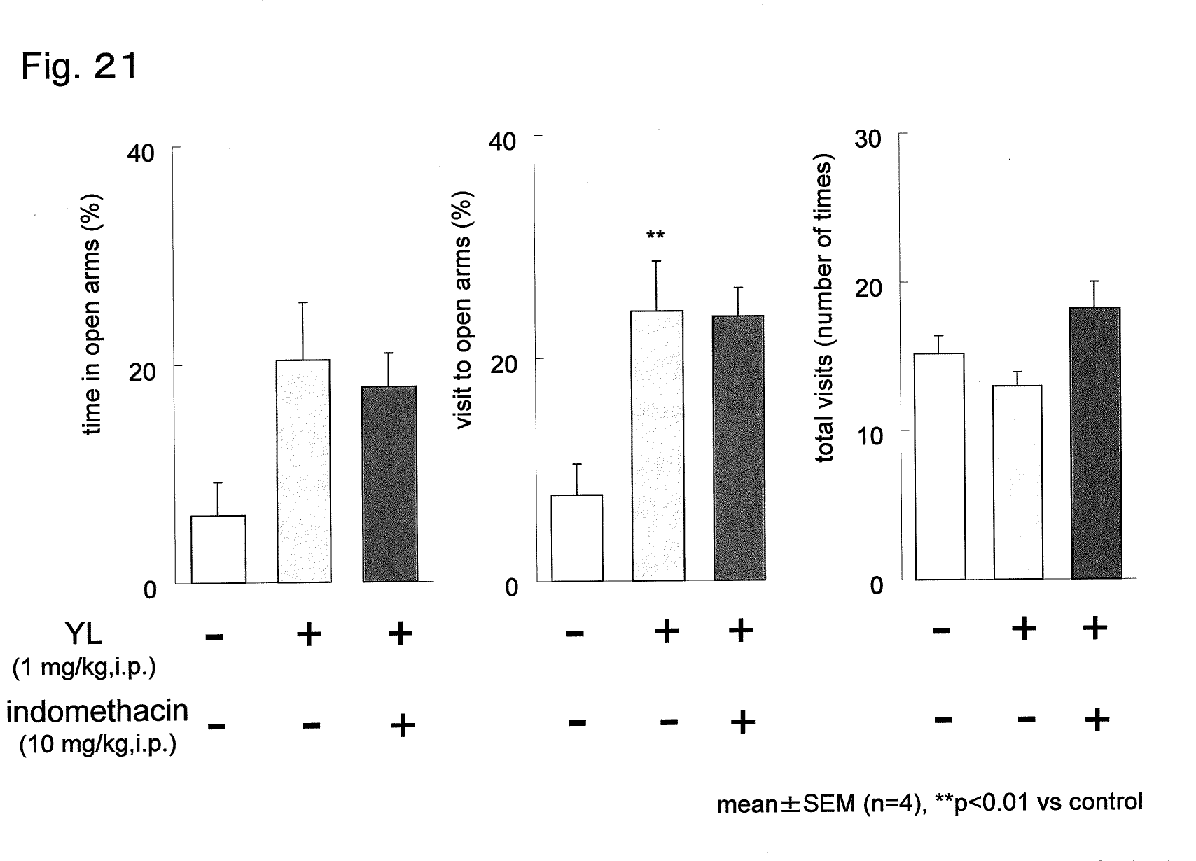

YL, an anxiolytic peptide of the invention, was administered in combination with the antagonists for various receptors listed in Table 2, and tests were conducted in the same manner as in Example 1 in order to identify the effect of each antagonist on the anxiolytic activity of YL; i.e., to identify the receptors on which the peptide of the invention acts. The results are shown in FIGS. 15 to 21, and Table 2.

TABLE 2
Effects of Various Antagonists on Anxiolytic Effects of YL

| | | Anxiolytic |
|---|---|---|
| Inhibitor Name | Inhibitor Properties | Effects of YL |
| WAY100135 | 5HT_{1A} antagonist | A |
| SCH23390 | dopamine D₁ antagonist | A |
| bicuculline | GABA_{A} antagonist | A |
| flumazenil | benzodiazepine antagonist | B |
| naloxone | μ opioid antagonist | C |
| naltrindole | δ opioid antagonist | C |
| BMY14802 | σ₁ antagonist | C |
| indomethacin | cyclooxygenase inhibitor | C |

A: Inhibited,
C: Uninhibited,
B: Partially inhibited

TABLE 3
Example 1, Anxiolytic Effects of YL in the Open-Field Test

| | cont. | YL (0.1 mg/kg, i.p.) |
|---|---|---|
| Time in Center Circle (%) | 0.889 ± 0.318 | 2.06 ± 0.234* |
| Visits to Center Circle (%) | 1.53 ± 0.428 | 3.02 ± 0.404* |
| Locomotor Activity | 187 ± 13.4 | 189 ± 22.2 |

mean ± SEM (n = 6).
*p < 0.05

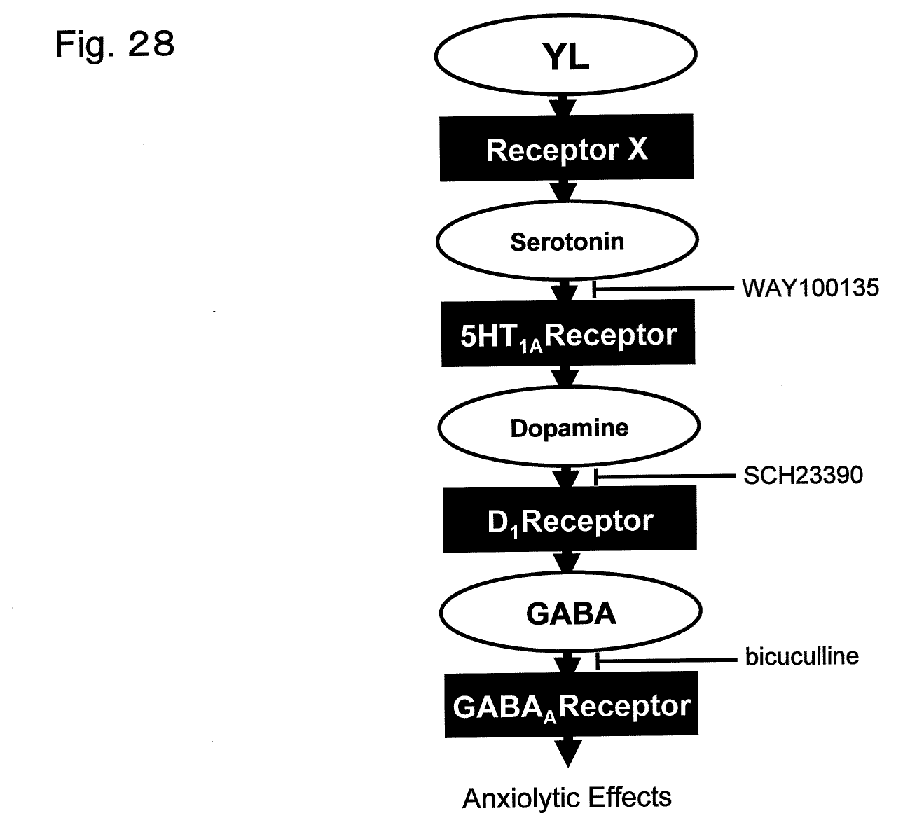

From the results shown in Table 2, and other experimental results (data not shown) showing the order of activation of serotonin, dopamine, and GABA, YL is presumed to have the anxiolytic pathway shown in FIG. 28.

Furthermore, the anxiolytic effects of YL were confirmed in both the elevated plus-maze experiment and the open-field test, establishing the usefulness of the peptides of the invention.

The anxiolytic drugs of the invention potentially have a mechanism of action different from those of the previous anxiolytic drugs, and thus can provide novel types of drugs.

1. A pharmaceutical or a pharmaceutical composition comprising, as an active ingredient, a peptide containing Tyr, hereinafter also abbreviated as Y, Phe, hereinafter also abbreviated as F, Trp, hereinafter also abbreviated as W, or His, hereinafter also abbreviated as H, and a hydrophobic amino acid adjacent thereto, or an analog thereof.

2. The pharmaceutical or the pharmaceutical composition according to claim 1, wherein the active ingredient is a peptide containing Tyr or Phe and a hydrophobic amino acid adjacent thereto, or an analog thereof.

3. The pharmaceutical or the pharmaceutical composition according to claim 1, wherein the active ingredient is YL, FL, WL, HL, YI, FI, YV, LY, LF, LW, IY, IF, (Y/F/W/H)L(Y/F/W), (Y/F/W/H)LQ, L(Y/F/W)L, or (Y/F/W/H)L(Y/F/W)EIAR, wherein, hereinafter, L represents Leu, I represents Ile, V represents Val, Q represents Gln, E represents Glu, A represents Ala, R represents Arg, (Y/F/W/H) represents H, W, Y, or F, and (Y/F/W) represents W, F, or Y.

4. The pharmaceutical or the pharmaceutical composition according to claim 3, wherein the active ingredient is YL, FL, WL, HL, YI, FI, FV, LY, LF, LW, IY, IF, YLY, YLQ, LYL, or YLYEIAR.

5. The pharmaceutical or the pharmaceutical composition according to claim 1, which is an anxiolytic drug, a sleep-inducing drug, a sleep-enhancing drug, a drug for treating schizophrenia, or an antidepressant drug.

6. An anxiolytic or sleep-enhancing foodstuff comprising a peptide containing Tyr, Phe, Trp, or His and a hydrophobic amino acid adjacent thereto, or an analog thereof.

7. The anxiolytic or sleep-enhancing foodstuff according to claim 6, comprising a peptide containing Tyr or Phe and a hydrophobic amino acid adjacent thereto, or an analog thereof.

8. The anxiolytic or sleep-enhancing foodstuff according to claim 6, comprising YL, FL, WL, HL, YI, FI, YV, LY, LF, LW, IY, IF, (Y/F/W/H)L(Y/F/W), (Y/F/W/H)LQ, L(Y/F/W)L, or (Y/F/W/H)L(Y/F/W)EIAR.

9. The anxiolytic or sleep-enhancing foodstuff according to claim 8, comprising YL, FL, WL, HL, YI, FI, FV, LY, LF, LW, IY, IF, YLY, YLQ, LYL, or YLYEIAR.

10. A method for relieving anxiety or enhancing sleep, comprising administering an effective amount of a peptide containing Tyr, Phe, Trp, or His and a hydrophobic amino acid adjacent thereto, or an analog thereof, to a subject in need thereof.

11. The pharmaceutical or the pharmaceutical composition according to claim 2, which is an anxiolytic drug, a sleep-inducing drug, a sleep-enhancing drug, a drug for treating schizophrenia, or an antidepressant drug.

12. The pharmaceutical or the pharmaceutical composition according to claim 3, which is an anxiolytic drug, a sleep-inducing drug, a sleep-enhancing drug, a drug for treating schizophrenia, or an antidepressant drug

13. The pharmaceutical or the pharmaceutical composition according to claim 4, which is an anxiolytic drug, a sleep-inducing drug, a sleep-enhancing drug, a drug for treating schizophrenia, or an antidepressant drug
